(19) United States
(12) Patent Application Publication
(10) Pub. No.: US 2003/0166631 A1
(43) Pub. Date: Sep. 4, 2003

(54) Pharmaceutical compositions and methods for administering EP2 receptor selective agonists

(76) Inventors: Francis Dumont, Ledyard, CT (US); Jinyang Hong, Stonington, CT (US); Yesook Kim, Branford, CT (US); Richard W. Korsmeyer, Old Lyme, CT (US); Mei Li, Westerly, RI (US); Vishwas M. Paralkar, Madison, CT (US); David D. Thompson, Gales Ferry, CT (US)

Correspondence Address: PFIZER INC., PATENT DEPARTMENT, MS8260-1611, EASTERN POINT ROAD, GROTON, CT, 06340, US

(21) Appl. No.: 10/305,649

(22) Filed: Nov. 26, 2002

Related U.S. Application Data
(60) Provisional application No. 60/335,156, filed on Nov. 30, 2001.

(51) Int. Cl. A61K 31/55; A61K 31/553; A61K 31/554; A61K 31/54; A61K 31/537; A61K 31/496; A61K 31/426; A61K 31/421

(52) U.S. Cl. 514/211.01; 514/478; 514/521; 514/603; 514/227.5; 514/255.03; 514/317; 514/237.8; 514/218; 514/217.12; 514/365; 514/374; 514/408; 514/400

(57) ABSTRACT

This invention is directed to pharmaceutical compositions and methods comprising prostglandin agonists, specifically EP₂ receptor selective agonists, which are useful to enhance bone repair and healing and restore or augment bone mass in vertebrates, particularly mammals. The EP₂ receptor selective agonists of the present invention are effective in the treatment of conditions such as those in which the patient has delayed or non-union fracture, bone defect, spinal fusion, bone in-growth, cranial facial reconstruction or bone sites at risk for fracture.

### CROSS REFERENCE TO RELATED APPLICATION

This application claims priority of U.S. provisional application No. 60/335,156, filed Nov. 30, 2001.

### BACKGROUND OF THE INVENTION

The present invention relates to pharmaceutical compositions and methods of administration of prostaglandin agonists, specifically EP₂ receptor selective agonists, which are useful to enhance bone repair and healing and restore or augment bone mass in vertebrates, particularly mammals. The EP₂ receptor selective agonists of the present invention are effective in the treatment of conditions such as those in which the patient has delayed or non-union fracture, bone defect, spinal fusion, bone in-growth, cranial facial reconstruction, and bone sites at risk for fracture.

The U.S. National Osteoporosis Foundation estimates that currently 25 million Americans are affected by osteoporosis and are at heightened risk for skeletal fractures. The number of women and men who will suffer from osteoporosis will rise as the worldwide population greater than 60 years of age increases from about 540 million to more than 1 billion by the year 2020. Approved therapies for the prevention and treatment of osteoporosis are unable to restore bone mass back to young adult levels. Current treatments are capable of only reducing fractures by about 50% and, thus, a high number of osteoporotic as well as non-osteoporotic fractures still occurs. Each year in the U.S., 7.9 million individuals suffer a skeletal fracture, of which 1.5 million are directly attributable to osteoporosis resulting in $13.8 billion in healthcare costs. Additionally, approximately 10% of fractures have delayed union, and about 1% of the total results in non-union requiring aggressive medical intervention to prevent long term disability. An average of 24% of hip fracture patients, age 50 and over, die in the year following their fracture. Therefore, improved therapies to treat skeletal fractures and insure bone union is needed.

Approximately 425,000 bone graft procedures are performed each year for closure of bone gaps. Of these procedures, about 50% are for spinal fusions including interbody fusion grafts and pedicular fixation. The remaining 50% is divided between delayed or non-union of fractures, hip fractures, total hip revision and tibial plateau fractures. The current gold standard of therapy for delayed union or nonunion fractures is the bone graft, a procedure where bone is harvested from the iliac crest and grafted into the injury site. While heal rates are high, there are considerable drawbacks, as the procedure results in pain at the site of harvest, extended operating time, increased blood loss, and heightened risk of infection. Autograft availability may also be limited by insufficient available tissue, especially in patients with osteoporosis or in patients who have undergone prior graft harvest. Allograft substitutes, such as demineralised bone material (DMB) are also commonly used, but these are also associated with risk of infection, inconsistent performance, limited supply and poor inductive ability. Treatments which would improve bone union in spinal fusion, fracture healing, reduce the need for bone grafting, and reduce the incidence of bone fracture non-unions would be expected to have significant medical benefits.

Prostaglandin E₂ (PGE₂) has been demonstrated to significantly increase bone mass when administered systemically or locally to the skeleton. However, due to severe side effects including diarrhea, lethargy, and flushing, PGE₂ is an unacceptable therapeutic option. It has been found that the EP-2 receptor subtype of PGE₂ receptor, and not EP-1 or EP-3, is responsible for the local bone anabolic activity of PGE₂ (see, e.g., Published International patent application, WO 98/27976) and that EP-1 and EP-3 receptor subtypes mediate some of the objectionable side effects.

Therefore, a selective EP-2 receptor agonist will increase bone formation and improve bone healing, but not possess PGE₂'s objectionable side effects. However, there is a need in the art for pharmaceutical compositions and methods of administration of selective EP-2 receptor agonists to promote bone formation and improve bone healing.

Published International patent applications WO 99/19300 and WO 98/28264 disclose prostaglandin agonists and their use to treat and promote the healing of bone fractures and osteotomies by local application (e.g., to the sites of bone fractures or osteotomies).

Abstract, “CP-463,755, A Non-prostanoid EP₂ Receptor Agonist, Stimulates Fracture Healing in a Rat Remoral Fracture Model,” American Society for Bone and Mineral Research (ASBMR) 2000, discloses that on days 3, 4 and 5 post-surgery, the rats were percutaneously injected with 0 or 5 mg of CP-463,755 to the fracture site. According to this abstract, the data demonstrated that CP-463,755 stimulated callus formation in rats.

S. C. Miller and S. C. Marks, Jr., Bone 14, 143-151 (1993), studied the local stimulation of new bone formation on the periosteal surface of the canine mandible by prostaglandin E₁ (PGE₁) and compared delivery by osmotic minipumps and controlled-release pellets implanted subperiosteally next to the lateral mandibular cortex.

S. C. Marks, Jr. and S. C. Miler, J. Oral Pathol. 17:500-505 (1988), reported that local infusion of PGE1 for 3 weeks at doses of 500 to 2000 μg per week produced a dramatic, localized formation of alveolar bone in the mandible of dogs.

In M-S. Shih and R. W. Norrdin, Am. J. Vet. Res. 48: 828-830 (1986), transverse fractures were made surgically in the ribs of adult beagles, and 0.5 ml of 10% ethanol Tris-buffer vehicle or 0.5 ml of PGE₁ (containing 0.2 mg of PGE₁ in 10% ethanol Tris-buffer) was injected directly into the fracture sites twice a day for 10 days. It was concluded that administration of PGE₁ induced bone matrix formation on the periosteal envelope adjacent to the fracture site and its contralateral matching site.

M-S. Shih and R. W. Norrdin, Calcif. Tissue Int. (1986) 39: 191-197, studied the effect of PGE₁ (0.2 mg/kg in 10% ethanol) injected into the defect site in the tibias of beagles twice a day for 10 days after surgery. It was found that the dogs that had received PGE₁ locally had more periosteal and cortical endosteal bone formation, with an increased amount of osteoid present.

R. Yang, T. Liu and S. Lin-Shiau, Calcif. Tissue Int., 52:57-61 (1993), investigated the effect of daily injections of prostaglandin E2 via the intraosseous route into the metaphysis of the left tibia for 14 days. According to this reference, this dosing regimen resulted in a significant increase of trabecular bone in the metaphysis.

K. Notoya et al., The Journal of Pharmacology and Experimental Therapeutics, 290: 1054-1064 (1999), examined the effect of TAK-778, a novel osteoblast differentiation promoting compound, in sustained-release microcapsules applied locally on skeletal regeneration and bone repair in vivo.

### SUMMARY OF THE INVENTION

The present invention provides the following:

A method for treating a bone fracture, bone injury or bone defect in a patient comprising local administration to the patient of a therapeutically effective amount of an EP₂ receptor selective agonist once a day for a period of about 7 days or greater.

More particularly, the present invention provides the above method wherein the agonist is administered once a day for about 7 to about 14 days. Even more particularly, the present invention provides the above method wherein the agonist is administered once a day for about 14 days. More particularly, the present invention provides the above method wherein the agonist is administered once a day for about 14 to about 21 days. More particularly, the present invention provides the above method wherein the agonist is administered once a day for about 14 to about 28 days.

More particularly, the present invention provides the above method wherein the therapeutically effective amount of the agonist is between about 0.001 to about 100 mg/kg/day. Even more particularly, the present invention provides the above method wherein the amount of the agonist is between about 0.01 to about 10 mg/kg/day.

More particularly, the present invention provides the above method wherein the agonist is administered by direct injection in a pharmaceutically acceptable buffer at or near the site where bone growth is needed. More particularly, the present invention provides the above method wherein the agonist is administered by direct injection in a pharmaceutically acceptable buffer at or near the site of the bone fracture, bone injury or bone defect. More particularly, the present invention provides such method wherein the agonist is administered by a catheter at or near the site where bone growth is needed.

In addition, the present invention provides a method for treating a bone fracture, bone injury or bone defect in a patient comprising local administration to the patient of a therapeutically effective amount of an EP₂ receptor selective agonist in a controlled release formulation;

wherein the agonist is administered in an oily suspension of an insoluble salt of the agonist;

wherein the agonist is administered in a bone glue formulation;

wherein the agonist is administered in a hydrophilic matrix containing poloxamers;

wherein the agonist is administered in controlled-release, biodegradable lipid vessicles;

wherein the agonist is administered in controlled-release, biodegradable poly(lactide-co-glycolide) microparticles;

wherein the agonist is administered in a polyanionic polysaccharide formulation;

wherein the agonist is administered in high viscosity liquid carrier material or lower viscosity liquid carrier material;

wherein the agonist is administered in carbonated apatite or hydroxyapatite formulation and a biocompatible source of calcium;

wherein the agonist is administered in collagen-containing carrier preparation; or

wherein the agonist is administered in formulations of thrombin, fibrin or synthetic peptides derived therefrom.

More particularly, the present invention provides the above method wherein the lipid vessicles are liposomes. More particularly, the present invention provides the above method wherein the polyanionic polysaccharide is hyaluronic acid or carboxymethylcellulose. More particularly, the present invention provides the above method wherein the high viscosity liquid carrier material is sucrose acetate isobutyrate.

More particularly, the present invention provides the above method wherein the agonist is released for a period of about 3 days or greater. Even more particularly, the present invention provides the above method wherein the agonist is released over a period of about 7 to about 28 days. Also, the present invention provides the above method wherein the agonist is released over a period of about 7 to about 14 days. More particularly, the present invention provides the above method wherein the agonist is released over a period of about 12 to about 14 days.

The present invention also provides the above method wherein the agonist is administered by direct injection at or near the site where bone growth is needed. More particularly, the present invention provides the above method wherein the agonist is administered by direct injection at or near the site of the bone fracture, bone injury or bone defect.

More particularly, the present invention provides the above methods wherein the EP₂ receptor selective agonist is a compound of Formula I or II, a prodrug thereof, or a pharmaceutically acceptable salt of the compound or the prodrug, wherein the variables are defined in the Detailed Description below.

In addition, the present invention provides a controlled release microparticle pharmaceutical composition for the sustained release of an EP₂ receptor selective agonist which comprises an EP₂ receptor selective agonist and a biocompatible, biodegradable poly(lactide-co-glycolide) polymer.

More particularly, the present invention provides the above composition wherein the EP₂ receptor selective agonist is a compound of Formula I or II, a prodrug thereof, or a pharmaceutically acceptable salt of the compound or the prodrug, wherein the variables are defined in the Detailed Description below.

More particularly, the present invention provides the above composition wherein the composition is locally administered at or near the site of the bone fracture, bone injury or bone defect. More particularly, the present invention provides the above composition wherein wherein the agonist is released over a period of about 7 to about 28 days.

The present invention is also directed to compositions and methods of treating a condition which presents with low bone mass in a mammal comprising administering to said mammal an EP₂ receptor selective agonist. According to the present invention, the compositions are administered locally. Conditions which present with low bone mass which are treated by the compositions and methods of this invention include, but are not limited to, osteoporosis, osteoporotic fractures, bone defects, childhood idiopathic bone loss, alveolar bone loss, mandibular bone loss, bone fracture, osteotomy, bone loss associated with periodontitis, prosthetic ingrowth and local bone rescue at skeletal sites that are at high risk of fracture in osteoporotic patients.

Preferably post-menopausal women and men over the age of 60 are treated. Also preferred is treatment of individuals, regardless of age, who have significantly reduced bone mass, i.e., greater than or equal to 1.5 standard deviations below young normal levels.

Methods for treating “secondary osteoporosis” are also included within the methods of this invention. “Secondary osteoporosis” includes glucocorticoid-induced osteoporosis, hyperthyroidism-induced osteoporosis, immobilization-induced osteoporosis, heparin-induced osteoporosis and immunosuppressive-induced osteoporosis in a vertebrate, e.g., a mammal (including a human being). Said treatment is achieved by administering to said vertebrate, e.g., a mammal, suffering from “secondary osteoporosis,” a “secondary osteoporosis” effective treating amount of a pharmaceutical composition comprising an EP₂ receptor selective agonist, a prodrug thereof or a pharmaceutically acceptable salt of said EP₂ receptor selective agonist or said prodrug.

Yet another aspect of this invention is directed to methods for strengthening a bone graft, inducing vertebral synostosis, enhancing long bone extension, enhancing bone healing following facial reconstruction, maxillary reconstruction or mandibular reconstruction in a vertebrate, e.g., a mammal (including a human being), comprising administering to said vertebrate, e.g., a mammal which has undergone facial reconstruction, maxillary reconstruction or mandibular reconstruction, a bone enhancing amount of a pharmaceutical composition comprising an EP₂ receptor selective agonist, a prodrug thereof or a pharmaceutically acceptable salt of said EP₂ receptor selective agonist or said prodrug.

The phrase “condition(s) which presents with low bone mass” refers to a condition where the level of bone mass is below the age specific normal as defined in standards by the World Health Organization “Assessment of Fracture Risk and its Application to Screening for Postmenopausal Osteoporosis (1994), Report of a World Health Organization Study Group, World Health Organization Technical Series 843”. Included in “condition(s) which presents with low bone mass” are primary and secondary osteoporosis. Secondary osteoporosis includes glucocorticoid-induced osteoporosis, hyperthyroidism-induced osteoporosis, immobilization-induced osteoporosis, heparin-induced osteoporosis and immunosuppressive-induced osteoporosis. Also included is periodontal disease, alveolar bone loss, post-osteotomy and childhood idiopathic bone loss. The phrase “condition(s) which presents with low bone mass” also includes long term complications of osteoporosis such as curvature of the spine, loss of height and prosthetic surgery.

The phrase “condition(s) which presents with low bone mass” also refers to a vertebrate, e.g., a mammal, known to have a significantly higher than average chance of developing such diseases as are described above including osteoporosis (e.g., post-menopausal women, and men over the age of 60).

Other bone mass augmenting or enhancing uses include bone restoration, increasing the bone fracture healing rate, replacing bone graft surgery entirely, enhancing the rate of successful bone grafts, bone healing following facial reconstruction, maxillary reconstruction, mandibular reconstruction, craniofacial reconstruction, prosthetic ingrowth, vertebral synostosis, long bone extension and spinal fusion.

The pharmaceutical compositions of the present invention may also be used in conjunction with orthopedic devices such as spinal fusion cages, spinal fusion hardware, internal and external bone fixation devices, screws and pins.

Those skilled in the art will recognize that the term bone mass actually refers to bone mass per unit area which is sometimes (although not strictly correctly) referred to as bone mineral density (BMD).

The term “treating”, “treat” or “treatment” as used herein includes preventative (e.g., prophylactic), palliative and curative treatment.

The term “effective amount” means an amount of a compound or combination of compounds that ameliorates, attenuates or eliminates a particular disease or condition or a symptom of a particular disease or condition, or prevents or delays the onset of a particular disease or condition or a symptom of a particular disease or condition.

The term “patient” means an animal, such as a human, a companion animal, such as a dog, cat and horse, and livestock, such as cattle, swine and sheep. Particularly preferred patients are mammals, including both males and females, with humans being even more preferred.

The term “pharmaceutically acceptable” as used herein means the carrier, vehicle, diluent, excipients and/or salt must be compatible with the other ingredients of the formulation, and not deleterious to the recipient thereof.

The expression “prodrug” refers to a compound that is a drug precursor which, following administration, releases the drug in vivo via some chemical or physiological process (e.g., a prodrug on being brought to the physiological pH or through enzyme action is converted to the desired drug form). Exemplary prodrugs upon cleavage release the corresponding drug compounds.

The expression “pharmaceutically acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine and tromethamine (2-amino-2-hydroxymethyl-1,3-propanediol).

The compositions and methods of this invention result in bone formation resulting in decreased fracture rates. This invention makes a significant contribution to the art by providing compositions and methods that increase bone formation resulting in prevention, retardation, and/or regression of osteoporosis and related bone disorders.

### DETAILED DESCRIPTION OF THE INVENTION

Any EP₂ receptor selective agonist may be used as the EP₂ receptor selective agonist of this invention. Preferred EP₂ receptor selective agonists include:

(i) compounds of Formula I

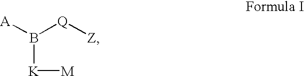

prodrugs thereof, and the pharmaceutically acceptable salts of the compounds and the prodrugs, wherein:

B is N;

A is (C₁-C₆)alkylsulfonyl, (C₃-C₇)cycloalkylsulfonyl, (C₃-C₇)cycloalkyl(C₁-C₆)alkylsulfonyl, said A moieties optionally mono-, di- or tri-substituted on carbon independently with hydroxy, (C₁-C₄)alkyl or halo;

Q is

—(C₂-C₆)alkylene-W—(C₁-C₃)alkylene-,

—(C₃-C₈)alkylene-, said —(C₃-C₈)alkylene- optionally substituted with up to four substituents independently selected from fluoro or (C₁-C₄)alkyl,

—X—(C₁-C₅)alkylene-,

—(C₁-C₅)alkylene-X—,

—(C₁-C₃)alkylene-X—(C₁-C₃)alkylene-,

—(C₂-C₄)alkylene-W-X—(C₀-C₃)alkylene-,

—(CO—C₄)alkylene-X-W—(C₁-C₃)alkylene-,

—(C₂-C₅)alkylene-W—X—W—(C₁-C₃)alkylene-, wherein the two occurrences of W are independent of each other,

—(C₁-C₄)alkylene-ethenylene-(C₁-C₄)alkylene-,

—(C₁-C₄)alkylene-ethenylene-(C₀-C₂)alkylene-X—(C₀-C₅)alkylene-,

—(C₁-C₄)alkylene-ethenylene-(CO—C₂)alkylene-X—W—(C₁-C₃)alkylene-,

—(C₁-C₄)alkylene-ethynylene-(C₁-C₄)alkylene-, or

—(C₁-C₄)alkylene-ethynylene-X—(C₀-C₃)alkylene-;

W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;

X is a five- or six-membered aromatic ring optionally having one or two heteroatoms selected independently from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;

Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;

K is a bond, (C₁-C₈)alkylene, thio(C₁-C₄)alkylene or oxy(C₁-C₄)alkylene, said (C₁-C₈)alkylene optionally mono-unsaturated and wherein K is optionally mono-, di- or tri-substituted independently with fluoro, methyl or chloro;

M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five- to eight-membered ring optionally having one to four heteroatoms selected independently from oxygen, sulfur and nitrogen, or, a bicyclic ring consisting of two fused partially saturated, fully saturated or fully unsaturated five- or six-membered rings, taken independently, optionally having one to four heteroatoms selected independently from nitrogen, sulfur and oxygen;

said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;

R¹, R² and R³ are optionally mono-, di- or tri-substituted on carbon independently with halo or hydroxy; and

V is a bond or (C₁-C₃)alkylene optionally mono- or di-substituted independently with hydroxy or fluoro;

(ii) compounds of Formula II

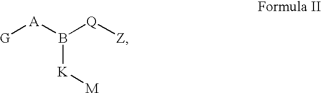

prodrugs thereof, and the pharmaceutically acceptable salts of the compounds and the prodrugs, wherein:

A is SO₂ or CO;

G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹ wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;

B is N or CH;

Q is

—(C₂-C₆)alkylene-W—(C₁-C₃)alkylene-, said alkylenes each optionally substituted with up to four substituents independently selected from fluoro or (C₁-C₄)alkyl,

—(C₄-C₈)alkylene-, said alkylene optionally substituted with up to four substituents independently selected from fluoro or (C₁-C₄)alkyl,

—X—(C₁-C₅)alkylene-, said alkylene optionally substituted with up to four substituents independently selected from fluoro or (C₁-C₄)alkyl,

—(C₁-C₅)alkylene-X—, said alkylene optionally substituted with up to four substituents independently selected from fluoro or (C₁-C₄)alkyl,

—(C₁-C₃)alkylene-X—(C₁-C₃)alkylene-, said alkylenes each optionally substituted with up to four substituents independently selected from fluoro or (C₁-C₄)alkyl,

—(C₂-C₄)alkylene-W-X—(C₀-C₃)alkylene-, said alkylenes each optionally substituted with up to four substituents each independently selected from fluoro or (C₁-C₄)alkyl,

—(C₀-C₄)alkylene-X-W—(C₁-C₃)alkylene-, said alkylenes each optionally substituted with up to four substituents each independently selected from fluoro or (C₁-C₄)alkyl,

—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,

—(C₁-C₄)alkylene-ethenylene-(C₁-C₄)alkylene-, said alkylenes and said ethenylene each optionally substituted with up to four substituents each independently selected from fluoro or (C₁-C₄)alkyl,

—(C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,

—(C₁-C₄)alkylene-ethenylene-(C₀-C₂)alkylene-X—W—(C₁-C₃)alkylene-, said alkylenes and said ethenylene each optionally substituted with up to four substituents each independently selected from fluoro or (C₁-C₄)alkyl,

—(C₁-C₄)alkylene-ethynylene-(C₁-C₄)alkylene-, said alkylenes and said ethynylene each optionally substituted with up to four substituents each independently selected from fluoro or (C₁-C₄)alkyl, or

—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;

Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;

K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;

M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;

Ar is a partially saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, taken independently, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five- to seven-membered ring having one or two heteroatoms selected independently from oxygen, sulfur and nitrogen;

Ar¹ and Ar² are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;

said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;

Ar³, Ar⁴ and Ar⁵ are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;

said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₀-C₆)alkylthio, (C₁-C₆)alkylsulfinyl, (C₁-C₄)alkylsulfonyl or mono-N— or di-N,N—(C₁-C₄)alkylaminosulfinyl;

W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;

X is a five- or six-membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethoxy, difluoromethoxy, hydroxyl, (C₁-C₄)alkoxy, or carbamoyl;

R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and

V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro.

(iii) compounds of Formula III

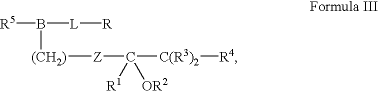

prodrugs thereof, and the pharmaceutically acceptable salts of the compounds and the prodrugs, wherein:

B is N or C(Q¹), where Q¹ is H or (C₁-C₃)alkyl;

L is n-propylenyl-X— or CH₂-metaphenylene-CH₂, wherein X is furanyl, thienyl, thiazolyl or tetrahydrofuranyl, said CH₂-metaphenylene-CH₂ or X being optionally

mono-, di- or tri-substituted on aromatic carbon independently with one to three chloro, fluoro, methoxy, difluoromethoxy, trifluoromethoxy, trifluoromethyl or methyl;

R is carboxyl, (C₁-C₆)alkoxycarbonyl, tetrazolyl, 5-oxo-1,2,4-thiadiazolyl; 5-oxo-1,2,4-oxadiazolyl, (C₁-C₄)alkylsulfonylcarbamoyl or phenylsulfonylcarbamoyl;

R¹ is H, methyl, ethyl or propyl;

R² is H or (C₂-C₅) alkanoyl;

R³ is independently H, fluoro or methyl;

R⁴ is H, (C₁-C₇) alkyl, or R⁴ and R¹ are taken together to form a 5-9 membered carbocyclic ring, said alkyl being optionally monounsaturated and optionally mono-, di- or tri-substituted independently with one to three fluoro, chloro, methoxy, difluoromethoxy, trifluoromethoxy, trifluoromethyl or methyl;

R⁵ is (C₁-C₆)alkylsulfonyl, (C₃-C₇)cycloalkylsulfonyl, (C₃-C₇)cycloalkyl(C₁-C₆)alkylsulfonyl, (C₁-C₆)alkylcarbonyl, (C₃-C₇)cycloalkylcarbonyl, (C₃-C₇)cycloalkyl(C₁-C₆)alkylcarbonyl, G-sulfonyl or G-carbonyl, said (C₁-C₆)alkylsulfonyl, (C₃-C₇)cycloalkylsulfonyl, (C₃-C₇)cycloalkyl(C₁-C₇)alkylsulfonyl, (C₁-C₆)alkylcarbonyl, (C₃-C₇)cycloalkylcarbonyl, (C₃-C₇)cycloalkyl(C₁-C₆)alkylcarbonyl optionally mono-, di- or tri-substituted on carbon independently with hydroxy, fluoro, chloro, methoxy, difluoromethoxy, trifluoromethoxy, trifluoromethyl or methyl;

Z is methylene, ethylene, propylene or ethenylene;

G is Ar, Ar¹—V—Ar², Ar—(C₁-C₆)alkylene, Ar—CONH—(C₁-C₆)alkylene, R¹²R¹³-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹² and R¹³ may be taken separately and are independently selected from H and (C₁-C₈)alkyl, or R¹² and R¹³ are taken together with the nitrogen atom to which they are attached to form a five- or six-membered azacycloalkyl, said azacycloalkyl optionally containing an oxygen atom and optionally substituted with up to two oxo, hydroxy, (C₁-C₄)alkyl, fluoro or chloro;

Ar is a partially saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven-membered ring having one or two heteroatoms selected independently from oxygen, sulfur and nitrogen;

Ar¹ and Ar² are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, taken independently, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;

said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety, independently selected from R⁴, R¹⁵ and R¹⁶ wherein R¹⁴, R¹⁵ and R¹⁶ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl; and

V is a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted, when V is not a bond, independently with hydroxy or fluoro; and

(iv) compounds of Formula IV

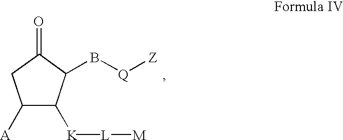

prodrugs thereof, and the pharmaceutically acceptable salts of the compounds and the prodrugs wherein:

A is hydrogen or hydroxy;

B is propylene, propenylene or propynylene;

Q is propylene, —CH₂OCH₂—, thiazolyl, pyridyl, phenyl or thienyl;

Z is carboxyl, (C₁-C₆)alkoxycarbonyl, tetrazolyl, 1,2,4-oxadiazolyl or 5-oxo-1,2,4-oxadiazolyl;

K is ethylene or ethenylene;

L is a bond or —CO—;

M is —Ar, —Ar¹—V—Ar², —Ar¹—S—Ar² or —Ar¹—O—Ar² wherein

Ar and Ar¹ are either

(1) each independently a fully unsaturated five- to eight-membered ring optionally having one to four heteroatoms selected independently from oxygen, sulfur and nitrogen, or a bicyclic ring consisting of two fused partially saturated, fully saturated or fully unsaturated five- and/or six-membered rings, taken independently, optionally having one to four heteroatoms selected independently from nitrogen, sulfur and oxygen, or a tricyclic ring consisting of three fused partially saturated, fully saturated or fully unsaturated five- and/or six-membered rings, taken independently, optionally having one to four heteroatoms selected independently from nitrogen, sulfur and oxygen, any of said partially saturated or fully saturated rings optionally having one or more oxo groups substituted on carbon, or

(2) each independently a fully saturated five to eight membered ring;

Ar² is a partially saturated, fully saturated or fully unsaturated five- to eight-membered ring optionally having one to four heteroatoms selected independently from oxygen, sulfur and nitrogen, or a bicyclic ring consisting of two fused partially saturated, fully saturated or fully unsaturated five- and/or six-membered rings, taken independently, optionally having one to four heteroatoms selected independently from nitrogen, sulfur and oxygen, or a tricyclic ring consisting of three fused partially saturated, fully saturated or fully unsaturated five- and/or six-membered rings, taken independently, optionally having one to four heteroatoms selected independently from nitrogen, sulfur and oxygen, any of said partially saturated or fully saturated rings optionally having one or more oxo groups substituted on carbon;

said Ar and Ar¹ moieties, when a fully unsaturated five- to eight-membered ring, a bicyclic ring or a tricyclic ring, and said Ar² moieties are each independently optionally substituted on carbon, on one ring if the moiety is monocyclic, on one or both rings if the moiety is bicyclic, or on one, two or three rings if the moiety is tricyclic, with up to three substituents selected from R¹, R² and R³ wherein R¹, R² and R³ are independently hydroxy, nitro, halo, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, aminocarbonylamino or mono-N—, di-N,N—, di-N,N′— or tri-N,N,N′—(C₁-C₄)alkyl substituted aminocarbonylamino, (C₁-C₄)alkanoylamino, (C₁-C₄)alkoxycarbonylamino, sulfonamido, hydroxysulfonyl, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;

R¹, R² and R³, when containing an alkyl, alkenyl, alkylene or alkenylene moiety, are optionally straight or branched and are optionally mono-, di- or tri-substituted on carbon independently with halo or hydroxy; and

V is a bond, —CO— or (C₁-C₃)alkylene optionally mono- or di-substituted independently with hydroxy or fluoro.

A preferred subgroup of Formula I compounds comprises those compounds selected from:

7-[(2′-hydroxymethyl-biphenyl-4-ylmethyl)-methanesulfonyl-amino]-heptanoic acid;

7-{[4-(3-hydroxymethyl-thiophen-2-yl)-benzyl]-methanesulfonyl-amino}-heptanoic acid;

7-[(2′-chloro-biphenyl-4-ylmethyl)-methanesulfonyl-amino]-heptanoic acid;

7-{[4-(1-hydroxy-hexyl)-benzyl]-methanesulfonyl-amino}-heptanoic acid;

7-[(4-butyl-benzyl)-methanesulfonyl-amino]-heptanoic acid;

7-{[5-(1-hydroxy-hexyl)-thiophen-2-ylmethyl]-methanesulfonyl-amino}-heptanoic acid;

(3-{[(4-butyl-benzyl)-methanesufonyl-amino]-methyl}-phenyl)-acetic acid;

7-{[3-(3-chloro-phenyl)-propyl]-methanesulfonyl-amino}-heptanoic acid;

7-{[3-(3,5-dichloro-phenyl)-propyl]-methanesufonyl-amino}-heptanoic acid;

5-(3-{[3-(3-chloro-phenyl)-propyl]-methanesulfonyl-amino}-propyl)-thiophene-2-carboxylic acid;

7-{[2-(3,5-dichloro-phenoxy)-ethyl]-methanesulfonyl-amino}-heptanoic acid;

5-(3-{[2-(3,5-dichloro-phenoxy)-ethyl]-methanesulfonyl-amino}-propyl)-thiophene-2-carboxylic acid;

N-[2-(3,5-dichloro-phenoxy)-ethyl]-N-[6-(1H-tetrazol-5-yl)-hexyl]-methanesulfonamide;

trans-(4-{[3-(3,5-dichloro-phenyl)-allyl]-methanesulfonyl-amino}-butoxy)-acetic acid;

trans-N-[3-(3,5-dichloro-phenyl)-allyl]-N-[6-(1H-tetrazol-5-yl)-hexyl]-methanesulfonamide;

trans-5-(3-{[3-(3,5-dichloro-phenyl)-allyl]-methanesulfonyl-amino}-propyl)-thiophene-2-carboxylic acid; and

trans-[3-({[3-(3,5-dichloro-phenyl)-allyl]-methanesulfonyl-amino}-methyl)-phenyl]-acetic acid; the prodrugs thereof, and the pharmaceutically acceptable salts of the compounds, and the prodrugs.

A preferred subgroup of Formula I compounds comprises those compounds selected from:

7-[(4-butyl-benzyl)-methanesulfonyl-amino]-heptanoic acid; and

7-{[2-(3,5-dichloro-phenoxy)-ethyl]-methanesulfonyl-amino}-heptanoic acid; or a pharmaceutically acceptable salt thereof.

A preferred subgroup of Formula II compounds comprises those compounds selected from:

(3-(((pyridine-3-sulfonyl)-(4-pyrimidin-5-yl-benzyl)-amino)-methyl)-phenyl)-acetic acid;

(3-(((5-phenyl-furan-2-ylmethyl)-(pyridine-3-sulfonyl)-amino)-methyl)-phenyl)-acetic acid;

(3-(((pyridine-3-sulfonyl)-(4-pyrimidin-2-yl-benzyl)-amino)-methyl)-phenyl)-acetic acid;

(3-(((pyridine-3-sulfonyl)-(4-thiazol-2-yl-benzyl)-amino)-methyl)-phenyl)-acetic acid;

(3-(((4-pyrazin-2-yl-benzyl)-(pyridine-3-sulfonyl)-amino)-methyl)-phenyl)-acetic acid;

(3-(((4-cyclohexyl-benzyl)-(pyridine-3-sulfonyl)-amino)-methyl)-phenoxy)-acetic acid;

(3-(((pyridine-3-sulfonyl)-(4-pyridin-2-yl-benzyl)-amino)-methyl)-phenoxy)-acetic acid;

(3-(((pyridine-3-sulfonyl)-(4-pyridin-3-yl-benzyl)-amino)-methyl)-phenoxy)-acetic acid;

(3-(((pyridine-3-sulfonyl)-(4-pyridin-4-yl)-benzyl)-amino)-methyl)-phenoxy)-acetic acid;

(3-(((pyridine-3-sulfonyl)-(4-thiazol-2-yl-benzyl)-amino)-methyl)-phenoxy)-acetic acid;

(3-(((2,3-dihydro-benzo[1,4]dioxin-6-ylmethyl)-(pyridine-3-sulfonyl)-amino)-methyl)-phenyl)-acetic acid;

(3-(((benzofuran-2-ylmethyl-(pyridine-3-sulfonyl)-amino)-methyl)-phenyl)-acetic acid;

(3-(((4-butyl-benzyl)-(pyridine-3-sulfonyl)-amino)-methyl)-phenyl)-acetic acid;

(3-(((benzenesulfonyl-(4-butyl-benzyl)-amino)-methyl)-phenyl)-acetic acid;

(3-(((4-butyl-benzyl)-(1-methyl-1H-imidazole-4-sulfonyl)-amino)-methyl)-phenyl)-acetic acid;

(3-(((4-dimethylamino-benzyl)-(pyridine-3-sulfonyl)-amino)-methyl)-phenyl)-acetic acid;

(3-(((4-dimethylamino-benzyl)-(pyridine-3-sulfonyl)-amino)-methyl)-phenoxy)-acetic acid;

(3-(((4-tert-butyl-benzyl)-(pyridine-3-sulfonyl)-amino)-methyl)-phenoxy)-acetic acid;

trans-(3-(((3-(3,5-dichloro-phenyl)-allyl)-(pyridine-3-sulfonyl)-amino)-methyl)-phenyl)-acetic acid; and

(3-(((2-(3,5-dichloro-phenoxy)-ethyl)-(pyridine-3-sulfonyl)-amino)-methyl)-phenoxy)-acetic acid; the prodrugs thereof, and the pharmaceutically acceptable salts of the compounds, and the prodrugs.

A preferred compound of Formula II is the sodium salt of (3-(((4-tert-butyl-benzyl)-(pyridine-3-sulfonyl)-amino)-methyl)-phenoxy)-acetic acid.

A preferred subgroup of Formula III compounds comprises compounds wherein:

B is N; R is carboxyl, (C₁-C₆)alkoxycarbonyl or tetrazolyl; Z is ethylenyl; R¹ and R² are each H; and L is CH₂-metaphenylene-CH₂ or n-propylene-X—; the prodrugs thereof, and the pharmaceutically acceptable salts of the compounds, and the prodrugs.

A further preferred subgroup of Formula III compounds comprises those compounds wherein:

R⁵ is selected from (C₁-C₆)alkycarbonyl, optionally mono-, di-, or tri-substituted with hydroxy or fluoro; (C₁-C₃)alkysulfonyl or (C₃-C₇)cycloalkysulfonyl; and G-sulfonyl, wherein G is phenyl, imidazolyl, pyridyl, pyrazolyl, or pyrimidyl optionally mono-, di-, or tri-substituted on carbon or nitrogen with chloro, fluoro, methoxy, difluoromethoxy, trifluoromethoxy, trifluoromethyl or methyl; the prodrugs thereof, and the pharmaceutically acceptable salts of the compounds, and the prodrugs.

A preferred subgroup of Formula IV compounds comprises those compounds selected from:

trans-7-(2-(2-(3,5-bis-trifluoromethyl-phenyl)-vinyl)-5-oxo-cyclopentyl)-heptanoic acid;

trans-7-(2-(2-(4-chloro-3-trifluoromethyl-phenyl)-vinyl)-5-oxo-cyclcopentyl)-heptanoic acid;

trans-7-(2-(2-(3,5-dichlorophenyl)-vinyl-5-oxo-cyclopentyl)-heptanoic acid;

trans-7-(2-(2-(3-chlorophenyl-vinyl)-5-oxo-cyclopentyl)-heptanoic acid;

trans-7-(2-oxo-5-(2-(3-trifluoromethyl-phenyl)-vinyl)-cyclopentyl)-heptanoic acid;

trans-7-(2-(2-(4-fluoro-phenyl)-vinyl)-5-oxo-cyclopentyl)-heptanoic acid;

ethyl trans-7-(2-(2-(3,5-bis-trifluoromethyl-phenyl)-vinyl)-5-oxocyclopentyl)-heptanoate;

ethyl trans-7-(2-(2-(4-chloro-3-trifluoromethyl-phenyl)-vinyl)-5-oxo-cyclopentyl)-heptanoate;

ethyl trans-7-(2-(2-(3,5-dichlorophenyl)-vinyl)-5-oxo-cyclopentyl)-heptanoate;

ethyl trans-7-(2-(2-(3-chlorophenyl)-vinyl)-5-oxo-cyclopentyl)-heptanoate;

ethyl trans-7-(2-oxo-5-(2-(3-trifluoromethyl-phenyl)-vinyl)-cyclopentyl)-heptanoate;

ethyl trans-7-(2-(2-(4-fluoro-phenyl)-vinyl)-5-oxo-cyclopentyl)-heptanoate;

trans-3-(2-(3,5-bis-trifluoromethyl-phenyl)-vinyl)—2-(6-(2H-tetrazol-5-yl)-hexyl)-cyclopentanone;

trans-3-(2-(4-chloro-3-trifluoromethylphenyl)-vinyl)-2-(6-(2H-tetrazol-5-yl)-hexyl)-cyclopentanone;

trans-3-(2-(3,5-dichloro-phenyl)-vinyl)-2-(6-(2H-tetrazol-5-yl)-hexyl)-cyclopentanone;

trans-3-(2-(3-chloro-phenyl)-vinyl)-2-(6-(2H-tetrazol-5-yl)-hexyl)-cyclopentanone;

trans-3-(2-(3-trifluoromethyl-phenyl)-vinyl)-2-(6-(2H-tetrazol-5-yl)-hexyl)-cyclopentanone; and

trans-3-(2-(4-fluoro-phenyl)-vinyl)-2-(6-(2H-tetrazol-5-yl)-hexyl)-cyclopentanone; the prodrugs thereof, and the pharmaceutically acceptable salts of the compounds, and the prodrugs.

The compounds of Formula I, the prodrugs thereof, and the pharmaceutically acceptable salts of the compounds and the prodrugs, may be prepared according to the synthetic methodologies described in Published International patent application WO 98/28264, which is incorporated by reference herein.

The compounds of Formula II, the prodrugs thereof, and the pharmaceutically acceptable salts of the compounds and the prodrugs, may be prepared according to the synthetic methodologies described in Published International patent application WO 99/19300, which is incorporated by reference herein.

The compounds of Formula III, the prodrugs thereof, and the pharmaceutically acceptable salts of the compounds and the prodrugs, may be prepared according to the synthetic methodologies described in published European patent application EP 0 911 321, which is incorporated by reference herein.

The compounds of Formula IV, the prodrugs thereof, and the pharmaceutically acceptable salts of the compounds and the prodrugs, may be prepared according to the synthetic methodologies described in published International patent application WO 98/58911, which is incorporated by reference herein.

Other EP₂ receptor selective agonists which may be used in the compositions and methods of this invention include compounds of the formula

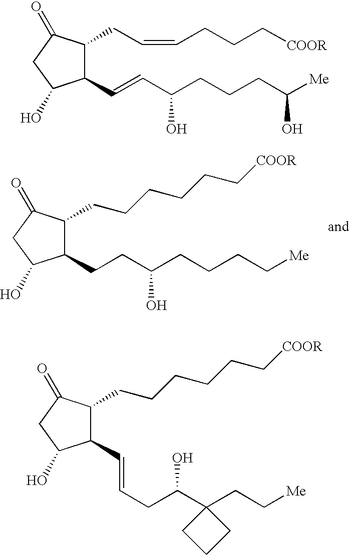

wherein the R is defined, and the compounds are prepared, as disclosed in U.S. Pat. No. 5,698,598, which is incorporated herein by reference.

Yet other EP₂ receptor selective agonists which may be used in the compositions and methods of this invention include compounds of the formula

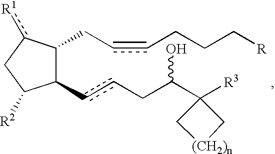

wherein the various substituents are defined, and the compounds are prepared, as disclosed in European Patent Application Publication No. EP 0 860 430, which is incorporated herein by reference.

Still other EP₂ receptor selective agonists which may be used in the compositions and methods of this invention include compounds of the formula

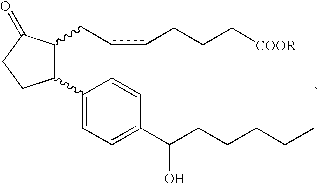

wherein the various substituents are defined, and the compounds are prepared, as disclosed in International Patent Application Publication No. WO95/19964, which is incorporated herein by reference.

Further EP₂ receptor selective agonists which may be used in the compositions and methods of this invention include compounds of the formula

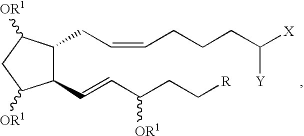

wherein the various substituents are defined, and the compounds are prepared, as disclosed in International Patent Application Publication No. WO99/25358, which is incorporated herein by reference.

More EP₂ receptor selective agonists which may be used in the compositions and methods of this invention include compounds of the formula

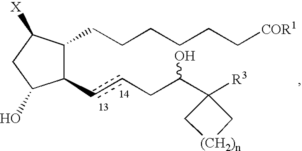

wherein the various substituents are defined, and the compounds are prepared, as disclosed in European Patent Application 0 974 580 and U.S. Pat. No. 6,235,780, which is incorporated herein by reference.

The compositions of this invention are all adapted to therapeutic use as agents that stimulate bone formation and increase bone mass in vertebrates, e.g., mammals, and particularly humans. Since bone formation is closely related to the development of osteoporosis and bone related disorders, these compositions, by virtue of their action on bone, prevent, arrest and/or regress osteoporosis. Also, these compositions would be useful to promote bone re-growth into skeletal areas where bone fractures, bone injuries or bone defects exist. For example, bone defects may be caused or produced by tumors in bone. Also, for example, these compositions would be useful to promote bone re-growth into skeletal areas where bone grafts are indicated.

The utility of the EP₂ receptor selective agonists and compositions thereof of the present invention as medical agents in the treatment of conditions which present with low bone mass (e.g., osteoporosis) and/or to treat bone fracture, bone injury or bone defects in vertebrates, e.g., mammals (especially humans and particularly female humans) is demonstrated by their activity in conventional in vitro assays, including a receptor binding assay and a cyclic AMP assay and in vivo assays, such as fracture healing assays (all of which are described below). Such assays also provide a means whereby the activities of the compositions of this invention can be compared to each other and with the activities of other known compounds and compositions. The results of these comparisons are useful for determining dosage levels in vertebrates, e.g., mammals, including humans, for the treatment of such diseases.

### Determination of cAMP Elevation in 293-S Cell Lines Stably Overexpressing Recombinant Human EP₂ Receptors

cDNAs representing the complete open reading frames of the human EP₂ receptors are generated by reverse transcriptase polymerase chain reaction using oligonucleotide primers based on published sequences (1, 2) and RNA from primary human kidney cells (EP₂) as templates. cDNAs are cloned into the multiple cloning site of pcDNA3 (Invitrogen Corporation, 3985B Sorrento Valley Blvd., San Diego, Calif. 92121) and used to transfect 293-S human embryonic kidney cells via calcium phosphate co-precipitation. G418-resistant colonies are expanded and tested for specific [³H]PGE₂ binding. Transfectants demonstrating high levels of specific [³H]PGE₂ binding are further characterized by Scatchard analysis to determine Bmax and Kds for PGE₂. The lines selected for compound screening have approximately 338,400 receptors per cell and a Kd=12 nM for PGE₂ (EP₂ receptor subtype). Constituitive expression of both receptors in parental 293-S cells is negligible. Cells are maintained in RPMI supplemented with fetal bovine serum (10% final) and G418 (700 ug/ml final).

cAMP responses in the 293-S/EP₂ are determined by detaching cells from culture flasks in 1 ml of Ca++ and Mg++ deficient PBS via vigorous pounding, adding serum-free RPMI to a final concentration of 1×10⁶ cells/ml, and adding 3-isobutyl-1-methylxanthine (IBMX) to a final concentration of 1 mM. One milliliter of cell suspension is immediately aliquoted into individual 2 ml screwcap microcentrifuge and incubated for 10 minutes, uncovered, at 37° C., 5% CO₂, 95% relative humdity. The compound to be tested is then added to cells at 1:100 dilutions such that final DMSO or ethanol concentrations is 1%. Immediately after adding compound, the tubes are covered, mixed by inverting two times, and incubated at 37° C. for 12 minutes. Samples are then lysed by incubation at 100° C. for 10 minutes and immediately cooled on ice for 5 minutes. Cellular debris is pelleted by centrifugation at 1000×g for 5 minutes, and cleared lysates are transferred to fresh tubes. cAMP concentrations are determined using a commercially available cAMP radioimmunoassay kit RIA (NEK-033, DuPont/NEN Research Products, 549 Albany St., Boston, Mass. 02118) after diluting cleared lysates 1:10 in cAMP RIA assay buffer (included in kit). Typically, one treats cells with 6-8 concentrations of the compound to be tested in 1 log increments. EC50 calculations are performed on a calculator using linear regression analysis on the linear portion of the dose response curves.

### REFERENCES

1. Regan, J. W. Bailey, T. J. Pepperl, D. J. Pierce, K. L. Bogardus, A. M. Donello, J. E. Fairbairn, C. E. Kedzie, K. M. Woodward, D. F. and Gil, D. W. 1994 Cloning of a Novel Human Prostaglandin Receptor with Characteristics of the Pharmaclogically Defined EP₂ Subtype. Mol. Pharmacology 46:213-220.

2. Bastien, L., Sawyer, N., Grygorczyk, R., Metters, K., and Adam, M. 1994 Cloning, Functional Expression, and Characterization of the Human Prostaglandin E2 Receptor EP₂ Subtype. J. Biol. Chem. Vol 269, 16:11873-11877.

### Assay for Binding to Prostaglandin E₂ Receptors

Membrane Preparation: All operations are performed at 4° C. Transfected cells expressing prostaglandin E₂ type 2 receptors (EP₂) are harvested and suspended to 2 million cells per ml in Buffer A (50 mM Tris-HCl (pH 7.4), 10 mM MgCl₂, 1 mM EDTA, 1 mM Pefabloc peptide, (Boehringer Mannheim Corp., Indianapolis, Ind.), 10 uM Phosporamidon peptide, (Sigma, St. Louis, Mo.), 1 uM pepstatin A peptide, (Sigma, St. Louis, Mo.), 10 uM elastatinal peptide, (Sigma, St. Louis, Mo.), 100 uM antipain peptide, (Sigma, St. Louis, Mo.)). The cells are lysed by sonification with a Branson Sonifier (Model #250, Branson Ultrasonics Corporation, Danbury, Conn.) in 2 fifteen second bursts. Unlysed cells and debris are removed by centrifugation at 100×g for 10 min. Membranes are then harvested by centrifugation at 45,000×g for 30 minutes. Pelleted membranes are resuspended to 3-10 mg protein per ml, protein concentration being determined by the method of Bradford (Bradford, M., Anal. Biochem., 72, 248 (1976)). Resuspended membranes are then stored frozen at −80° C. until use.

Binding Assay: Frozen membranes prepared as above are thawed and diluted to 1 mg protein per ml in Buffer A above. One volume of membrane preparation is combined with 0.05 volume test compound or buffer and one volume of 3 nM ³H-prostaglandin E₂ (#TRK 431, Amersham, Arlington Heights, Ill.) in Buffer A. The mixture (205 μL total volume) is incubated for 1 hour at 25° C. The membranes are then recovered by filtration through type GF/C glass fiber filters (#1205-401, Wallac, Gaithersburg, Md.) using a Tomtec harvester (Model Mach 11/96, Tomtec, Orange, Conn.). The membranes with bound ³H-prostaglandin E₂ are trapped by the filter, while the buffer and unbound ³H-prostaglandin E₂ pass through the filter into waste. Each sample is then washed 3 times with 3 ml of (50 mM Tris-HCl (pH 7.4), 10 mM MgCl₂, 1 mM EDTA). The filters are then dried by heating in a microwave oven. To determine the amount of ³H-prostaglandin bound to the membranes, the dried filters are placed into plastic bags with scintillation fluid and counted in a LKB 1205 Betaplate reader (Wallac, Gaithersburg, Md.). IC50s are determined from the concentration of test compound required to displace 50% of the specifically bound ³H-prostaglandin E₂.

The full length EP₂ receptor is made as disclosed in Regan et al., Molecular Pharmacology, 1994, 46, 213-220. This full length receptor is used to prepare 293S cells expressing the EP₂ receptors.

293S cells expressing the human EP₂ prostaglandin E₂ receptors are generated according to methods known to those skilled in the art. Typically, PCR (polymerase chain reaction) primers corresponding to the 5′ and 3′ ends of the published full length receptor are made according to the well known methods disclosed above and are used in an RT-PCR reaction using the total RNA from human lung (for EP₂) as a source. PCR products are cloned by the TA overhang method into pCR2.1 (Invitrogen, Carlsbad, Calif.) and identity of the cloned receptor is confirmed by DNA sequencing.

293S cells (Mayo, Dept. of Biochemistry, Northwestern Univ.) are transfected with the cloned receptor in pcDNA3 by electroporation. Stable cell lines expressing the receptor are established following selection of transfected cells with G418.

Clonal cell lines expressing the maximal number of receptors are chosen following a whole cell ³H-PGE₂ binding assay using unlabeled PGE₂ as a competitor.

### Fracture Healing Assays

### Assays for Effects on Fracture Healing after Local or Systemic Administration in Small Animals

Sprague-Dawley rats at 3 months of age are anesthetized with Ketamine. A 1 cm incision is made on the anteromedial aspect of the proximal part of the right tibia.

The following describes the tibial fracture technique: The incision is carried through to the bone, and a 1 mm hole is drilled 4 mm proximal to the distal aspect of the tibial tuberosity 2 mm medial to the anterior ridge. Intramedullary nailing is performed with a 0.8 mm stainless steel tube (maximum load 36.3 N, maximum stiffness 61.8 N/mm, tested under the same conditions as the bones). No reaming of the medullary canal is performed. A standardized closed fracture is produced 2 mm above the tibiofibular junction by three-point bending using specially designed adjustable forceps with blunt jaws. To minimize soft tissue damage, care is taken not to displace the fracture. The skin is closed with monofilament nylon sutures.

The following describes the femoral fracture technique in rats: Sprague-Dawley rats at 3 months of age are anesthetized with Ketamine and Xylazine at doses of 100 and 10 mg/kg, respectively. A 1 cm incision is made just lateral to the patella and the patella is pushed laterally to expose the femoral condyles. A Kirschner wire (0.045″ in diameter) is introduced into the intramedullary canal through the intercondylar portion. The Kirschner wire does not protrude into the knee joint or interfere with the motion of the patella. The skin incision is closed. The mid-diaphysis of the pinned femur is fractured by means of a three-point bending device driven by a dropped weight. The operation is performed under sterile conditions. Radiographs of all fractures are taken immediately after nailing, and rats with fractures outside the specified diaphyseal area or with displaced nails are excluded. The remaining animals are divided randomly into the following groups with 10 to 15 animals per each subgroup per time point for testing the fracture healing: One group of animals receives daily treatment with vehicle, while the others receive daily treatment of compounds at various doses by local injection into the fracture site or by systemic administration (oral, sc., iv etc.) for 10 to 80 days.

At various time points during the treatment period, 10-15 rats from each group are anesthetized with Ketamine and sacrificed by exsanguination. Both tibiofibular or femoral bones are removed by dissection and all soft tissue is stripped. All bones are X-rayed. Bone samples are further processed for biomechanical testing or histological testing.

Histological Analysis: The methods for histologic analysis of fractured bone have been previously published by Mosekilde and Bak (The Effects of Growth Hormone on Fracture Healing in Rats: A Histological Description, Bone, 14:19-27, 1993). Briefly, the fracture side is sawed 8 mm to each side of the fracture line, embedded undecalcified in methymethacrylate, and cut frontal sections on a Reichert-Jung Polycut microtome 8 μm thick. Masson-Trichrome stained mid-frontal sections (including both tibia and fibula) are used for visualization of the cellullar and tissue response to fracture healing with and without treatment. Sirius red stained sections are used to demonstrate the characterisitics of the callus structure and to differentiate between woven bone and lamellar bone at the fracture site. The following measurements are performed: (1) fracture gap—measured as the shortest distance between the cortical bone ends in the fracture, (2) callus length and callus diameter, (3) total bone volume area of callus, (4) bony tissue per tissue area inside the callus area, (5) fibrous tissue in the callus, and (6) cartilage area in the callus.

Biomechanical Analysis: The methods for biomechanical analysis have been previously published by Bak and Andreassen (The Effects of Aging on Fracture Healing in Rats, Calcif Tissue Int 45:292-297, 1989). Briefly, radiographs of all fractures are taken prior to the biomechanical test. The mechanical properties of the healing fractures are analyzed by a destructive three- or four-point bending or torsional procedure. Maximum load, stiffness, energy at maximum load, deflection at maximum load and maximum stress are determined.

### Assay for Effects on Fracture Healing after Local or Systemic Administration in Large Animals

Fracture Technique: Female or male beagle dogs at approximately 2 years of age are used under anesthesia in the study. Transverse radial fractures are produced by slow continuous loading in three-point bending as described by Lenehan et al. (Lenehan, T. M.; Balligand, M.; Nunamaker, D. M.; Wood, F. E.: Effects of EHDP on Fracture Healing in Dogs. J Orthop Res 3:499-507; 1985). The wire is pulled through the fracture site to ensure complete anatomical disruption of the bone. Thereafter, local delivery of prostaglandin agonists to the fracture site is achieved by daily injection into the fracture site, by slow release of compound delivered by slow release pellets, by administration of the compounds in a suitable formulation such as a paste gel solution or suspension or by systemic administration (e.g., oral, s.c., i.m. or i.v.) for 10, 15, or 20 weeks.

Histological Analysis: The methods for histologic analysis of fractured bone have been previously published by Peter et al. (Peter, C. P.; Cook, W. O.; Nunamaker, D. M.; Provost, M. T.; Seedor, J. G.; Rodan, G. A. Effects of alendronate on fracture healing and bone remodeling in dogs, J. Orthop. Res. 14:74-70, 1996) and Mosekilde and Bak (The Effects of Growth Hormone on Fracture Healing in Rats: A Histological Description, Bone, 14:19-27, 1993). Briefly, after sacrifice, the fracture side is sawed 3 cm to each side of the fracture line, embedded undecalcified in methymethacrylate, and cut on a Reichert-Jung Polycut microtome in 8 μm thick frontal sections. Masson-Trichrome stained mid-frontal sections (including both tibia and fibula) are used for visualization of the cellullar and tissue response to fracture healing with and without treatment. Sirius red stained sections are used to demonstrate the characteristics of the callus structure and to differentiate between woven bone and lamellar bone at the fracture site. The following measurements are performed: (1) fracture gap—measured as the shortest distance between the cortical bone ends in the fracture, (2) callus length and callus diameter, (3) total bone volume area of callus, (4) bony tissue per tissue area inside the callus area, (5) fibrous tissue in the callus, (6) cartilage area in the callus.

Biomechanical Analysis: The methods for biomechanical analysis have been previously published by Bak and Andreassen (The Effects of Aging on Fracture Healing in Rats, Calcif Tissue Int 45:292-297, 1989) and Peter et al. (Peter, C. P.; Cook, W. O.; Nunamaker, D. M.; Provost, M. T.; Seedor, J. G.; Rodan, G. A. Effects of Alendronate On Fracture Healing And Bone Remodeling In Dogs, J. Orthop. Res. 14:74-70, 1996). Briefly, radiographs of all fractures are taken prior to the biomechanical test. The mechanical properties of the healing fractures are analyzed by a destructive three- or four-point bending procedures. Maximum load, stiffness, energy at maximum load, deflection at maximum load, and maximum stress are determined.

### Combination and Sequential Treatment Protocol

The term “Second Active Agent” hereinafter refers collectively to pharmaceutical compounds or agents that are useful to treat fracture healing, bone repair and/or osteoporosis, a prodrug of said compounds or agents, or a pharmaceutically acceptable salt of such compound, agent or prodrug. Use of the term in singular form, as in “a Second Active Agent” hereinafter refers to a pharmaceutical agent selected from said Second Active Agents. A Second Active Agent may be a pharmaceutical agent that shares more than one of the foregoing characteristics.

An additional aspect of this invention relates to pharmaceutical compositions comprising an EP₂ receptor selective agonist of the present invention, and a Second Active Agent. Such compositions are hereinafter referred to collectively as the “combination compositions”.

This invention also relates to therapeutic methods for treating fracture healing bone injury or defect, bone repair and/or osteoporosis in a mammal wherein an EP₂ receptor selective agonist of the present invention and a Second Active Agent are administered together as part of the same pharmaceutical composition or separately. Such methods are hereinafter referred to collectively as the “combination therapies” of the present invention. Combination therapies include therapeutic methods wherein an EP₂ receptor selective agonist of the present invention and a Second Active Agent are administered together as part of the same pharmaceutical composition and to methods wherein these two agents are administered separately, either simultaneously or sequentially in any order.

This invention further provides pharmaceutical kits comprising an EP₂ receptor selective agonist of the present invention and a Second Active Agent. Such kits may hereinafter be referred to as the “kits” of the present invention.

Any anabolic agent, growth hormone, growth hormone secretagogue, bone morphogenic protein (BMP), parathyroid hormone (PTH), and an anti-resorptive agent, such as lasofoxifene, may be used as the Second Active Agent in the combination compositions, combination therapies and kits of the present invention.

The following protocols can of course be varied by those skilled in the art. For example, intact male or female rats or dogs, or sex hormone deficient male (orchidectomy) or female (ovariectomy) rats may be used. In addition, male or female rats at different ages (such as 12 months of age) can be used in the studies. The animals can be either intact or castrated (ovariectomized or orchidectomized), and locally administered with EP₂ receptor selective agonists such as the compounds of the present invention at different doses (such as 1, 3 or 6 mg/kg/day) for a certain period (such as a few days or 60 days), and followed by systemic administration of a Second Active Agent at different doses (such as 1, 5, 10 mg/kg/day) for a certain period (such as two weeks to two months), or combination treatment with both a local EP₂ receptor selective agonist and a systemic Second Active Agent at different doses for a certain period (such as two weeks to two months). In the castrated rats, treatment can be started on the next day after surgery (for the purpose of preventing bone loss) or at the time bone loss has already occurred (for the purpose of restoring bone mass). The rats are sacrificed under ketamine anesthesia. The similar endpoints are determined as described above in the Fracture Healing Assays.

Administration of the pharmaceutical compositions of the present invention of an EP₂ receptor selective agonist, a prodrug thereof or a pharmaceutically acceptable salt of said agonist or said prodrug can be via any method which delivers the composition of this invention locally (e.g., at the site of the bone fracture, osteotomy or orthopedic surgery). These methods include percutaneous, parenteral and other routes of administration during a closed surgical procedure or direct local application during an open surgical procedure.

The compounds of the present invention may be administered parenterally (e.g., intravenous, intramuscular, transdermal, subcutaneous, rectal or intramedullary injection). The compounds of the present invention may also be administered topically, for example, to an open wound.

The pharmaceutical compositions of the present invention can be used for the treatment and promotion of healing of bone fractures, bone injuries or bone defects and osteotomies by local administration or application (e.g., to the sites of bone fractures, injuries, defects or osteotomies) of the compositions of this invention. Local administration or application includes, e.g., direct injection through the skin, direct application during surgery, implant, cathether and other means available in the art. Local administration indicates that the concentration of the agonist at the site of administration is enhanced relative to the concentration of the agonist circulating in the body of the patient.

The compositions of the present invention are applied to the sites of bone fractures, bone injuries or bone defects, for example, either by injection of the compound in a suitable solvent (e.g., an oily solvent such as arachis oil) at or near the site of the bone fracture, bone injury or bone defect (including at the site of the bone fracture, bone injury or bone defect and/or close proximity to the site of the bone fracture, bone injury or bone defect), or, in cases of open surgery, by local application thereto of such compositions in a suitable vehicle, carrier or diluent such as bone-wax, demineralized bone powder, polymeric bone cements, bone sealants, etc. Alternatively, local application can be achieved by applying a solution or dispersion of the composition in a suitable carrier or diluent onto the surface of, or incorporating it into solid or semi-solid implants conventionally used in orthopedic surgery, such as dacron-mesh, gel-foam and kiel bone, or prostheses.

A therapeutically effective amount for the bone growth treatment for the EP-2 receptor selective agonists of the present invention range between about 0.001 to about 100 mg/kg/day, with an especially preferred amount being about 0.01 to about 10 mg/kg/day.

In any event, the amount and timing of compositions administered will, of course, be dependent on the subject being treated, on the severity of the affliction, on the manner of administration and on the judgment of the prescribing physician. Thus, because of patient to patient variability, the dosages given above are a guideline and the physician may titrate doses of the active compounds to achieve the treatment (e.g., bone mass augmentation) that the physician considers appropriate for the patient. In considering the degree of treatment desired, the physician must balance a variety of factors such as bone mass starting level, age of the patient, presence of preexisting disease, as well as presence of other diseases (e.g., cardiovascular disease).

There are many patients who would benefit from treatment according to the methods of the present invention, for example, a patient who has broken his/her hip and has surgery to repair it. The compositions of the present invention would enhance the fracture healing in the surgically repaired hip and could also be used to strengthen the patient's other hip, which may be weakened by, e.g., osteoporosis. In such circumstances, the compositions of the present invention would be administered locally to the patient's surgically repaired hip and other compositions, such as oral formulations, would be administered systemically to treat the patient's osteoporosis.

The EP₂ receptor selective agonists used in the compositions and methods of the present invention are generally administered in the form of a pharmaceutical composition comprising at least one of the compounds of this invention together with a pharmaceutically acceptable vehicle or diluent. Thus, the compounds of this invention can be administered individually or together in any conventional form such as parenteral, rectal or transdermal dosage form.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous, intraperitoneal and intramedullary injection, especially at or near the fracture site. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of an active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

Pharmaceutical compositions of the present invention may contain a total of 0.1%-95% of an EP₂ receptor selective agonist used in this invention, preferably 1%-70%. In any event, the composition or formulation to be administered will contain a quantity of the EP₂ receptor selective agonist in an amount effective to treat the disease/condition of the subject being treated, e.g., a bone fracture.

The EP₂ receptor selective agonist may be formulated for administration to a mammal by dissolving in an appropriate buffer such as 2% glycine or another pharmaceutically acceptable buffer, such as saline, 5% ethanol or other pharmaceutically acceptable alcohols, 20% β-cyclodextrin and others known in the art, taking care that the pH and tonicity of the resulting solution are within limits acceptable for injection, as known to those skilled in the art. In general, administration of such simple solutions by injection results in rapid absorption of the agonist from the injection site.

In addition to the simple, rapidly-absorbed solutions described above, the EP₂ receptor selective agonist may be formulated into sustained-release formulations for injection. Several such formulation approaches are described in Sustained-Release Injectable Products, eds. J. Senior and M. Radomsky (Denver, Colo.: Interpharm Press, 2000). These formulation approaches include the use of oily formulations, liposomes, polymeric microspheres, injectable hydrogels, and solidifying injections. These formulation approaches result in sustained absorption of the agonist from a localized depot. Formulations prepared by these approaches can retain the agonist within the depot, releasing it gradually over a period of time. These formulations achieve this prolonged release by various mechanisms, including physical partitioning, diffusion of the agonist from the formulation matrix, gradual erosion and dissolution of the formulation matrix itself. Some of these formulations may require single or multiple injections over a period of time, depending on the specific agonist being administered. Also, these formulations may be modified, using procedures available in the art, for specific applications or uses. In addition, initial administration of the formulations several days after the initial bone fracture, bone injury or bone defect or treatment therefor may be preferred. The ingredients in these formulations are commercially available or readily prepared according to literature procedures.

For example, an oily or aqueous suspension of the agonist or its insoluble salt will tend to remain as a depot after injection, releasing the agonist gradually as the agonist partitions between the oily phase of the depot and the aqueous phase of the body. Examples of such oils include sesame oil or peanut oil. Examples of insoluble salts include sodium, potassium, calcium, magnesium, benzathine, benethamine.

In another example, if the agonist is formulated into a hydrophilic matrix, such as poloxamer, after injection the agonist will slowly diffuse from the viscous poloxamer depot into the surrounding body fluid. In another example, if the agonist is encapsulated within lipid vessicles, such as liposomes, then it will be released at the injection site by gradual diffusion through the lipid layers of the liposomes, as well as by degradation of the liposomes. In another example, if the agonist is formulated in solid microparticles, such as microspheres, of poly(lactide-co-glycolide) (PLGH), the agonist will slowly diffuse from the solid microspheres. The PLGH microspheres will also degrade by hydrolysis in the aqueous body environment, releasing any portion of the agonist, which remains and eventually disappearing. Methods for the preparation of PLGH microspheres are known in the art, such as in M. Radomsky, L. Liu and T. Iwamoto, “Synthetic Polymers for Nanosphere and Microsphere Products,” in Sustained-Release Injectable Products, eds. J. Senior and M. Radomsky (Denver, Colo.: Interpharm Press, 2000), pp. 181-202, which is hereby incorporated by reference herein.

The following provides additional descriptions and examples of the sustained-release formulations of the EP₂ receptor selective agonists of the present invention:

The present invention relates to the use of poloxamers for sustained release of locally injected EP₂ agonists. Poloxamers are block copolymers of poly(ethylene oxide) and poly(propylene oxide). Poly(ethylene oxide) is typically present in the copolymer at levels of 10 to 80% by weight, preferably 60-80%. The poloxamer molecular weight ranges from 1,000 to 30,000, preferably 10,000 to 20,000. Very high molecular weight poloxamers are preferred. The poloxamer should be dissolved in an aqueous vehicle at concentrations ranging from 10-40% by weight, preferably 20-30%. The preferred vehicle is water. Alternative vehicles include physiologically compatible buffers, preferably at a concentration of 5-10 mM with a pH of 7 to 9. As used herein the term “EP₂ agonist” refers to the free acid form of a prostaglandin-E₂ receptor selective agonist or any of its salts, including for example the sodium salt. The concentration of EP₂ agonist in the vehicle can range from about 1 to about 200 mg/mL, preferably about 5 to about 150 mg/mL, even more preferably about 5 to about 50 mg/mL.

### EXAMPLE 1

Dissolve 2.5 g of poloxamer 407, MW 12,600 (brand name Pluronic® F127, BASF chemicals) in 7.5 g of water by stirring. Add 0.5 g of EP₂ agonist and stir to suspend or dissolve.

### EXAMPLE 2

Dissolve 2.0 g of poloxamer 338, MW 14,600 (brand name Pluronic® F108, BASF chemicals) in 8.0 g of water by stirring. Add 1.0 g of EP₂ agonist and stir to suspend or dissolve.

In addition, the present invention relates to the use of polyanionic polysaccharides for sustained release of locally injected EP₂ agonists. Preferred polyanionic polysaccharides for use in the methods of the present invention include hyaluronic acid (HA), carboxymethylcellulose (CMC), carboxymethyl amylose (CMA), chondroitin-6-sulfate, dermatin sulfate, heparin, and heparin sulfate or combinations thereof. HA is particularly preferred (see, e.g., published International patent application, WO 97/32591, which is incorporated by reference herein, for methods of promoting bone growth with hyaluronic acid and growth factors). As used herein the term “HA” means hyaluronic acid and any of its hyaluronic derivatives or salts, including for example, sodium hyaluronate. The polyanionic polysaccharide can be dissolved in solvents including water or physiologically compatible buffers. Preferred solvents are 5-50 mM phosphate or citrate buffers in the pH range of 3-8. The preferred concentration of polyanionic polysaccharide in the solvent is about 1 to about 10% (w/w), more preferably about 2% to about 7% (w/w). As used herein the term “EP₂ agonist” refers to the free acid form of a prostaglandin-E₂ receptor selective agonist or any of its salts, including for example the sodium salt. The EP₂ agonist should be dissolved in the polyanionic polysaccharide vehicle at a concentration about 1 to about 200 mg/mL, preferably about 5 to about 150 mg/mL, even more preferably about 5 to about 50 mg/mL. When EP₂ agonists are administered in polyanionic polysaccharide vehicles, such as hyaluronic acid or CMC, multiple doses of such formulations may be required for optimal results. Also, initial administration of the formulations several days after the initial bone fracture, bone injury or bone defect may be preferred.

### EXAMPLE 3

Dissolve 0.2 g of HA in 9.8 g of 10 mM, pH 4 citrate buffer by agitation. Add 0.5 g of EP₂ agonist, free acid and suspend in the vehicle by stirring.

### EXAMPLE 4

Dissolve 0.2 g of HA in 9.8 g of 25 mM, pH 7.4 phosphate buffer by agitation. Add 0.5 g of EP₂ agonist, sodium salt and dissolve in the vehicle by stirring.

Furthermore, the present invention relates to the use of a high viscosity liquid carrier material (HVLCM) for sustained release of locally injected EP₂ agonists. In one embodiment, the HVLCM is mixed with a viscosity lowering water soluble or miscible solvent such as ethanol, dimethylsulfoxide, ethyl lactate, ethyl acetate, benzyl alcohol, triacetin, N-methylpyrrolidone, propylene carbonate, glycofurol, freons, dimethyl ether, propane, butane, dimethyl formamide, dimethylacetamide, diethylene carbonate, butylene glycol, N-(betahydromethyl)lactamide, diokolanes and other amides, esters, ethers or alcohols to form a lower viscosity liquid carrier material (LVLCM). The preferred solvent is ethanol. The HVLCM can be stearate esters, stearate amides and other long-chain fatty acid amides, long-chain fatty alcohols or long-chain esters. The preferred HVLCM is sucrose acetate isobutyrate (SAIB), a sucrose molecule esterified with two acetic acid and six isobutyric acid moieties. The HVLCM is typically present in controlled delivery compositions in an amount in the range from 10-95% by weight, more typically, between 80-95% by weight. The composition optionally includes additives that modify the properties of the composition as desired. Non-limiting examples of suitable additives include biodegradable polymers, non-biodegradable polymers, natural or synthetic oils, carbohydrates or carbohydrate derivatives, BSA (bovine serum albumin), inorganic salts, surfactants and organic compounds such as sugars, and organic salts such as sodium citrate. As used herein the term “EP₂ agonist” refers to the free acid form of a prostaglandin-E₂ receptor selective agonist or any of its salts, including for example the sodium salt. The EP₂ agonist should be dissolved in the LVLCM vehicle at a concentration of about 1 to about 200 mg/mL, preferably about 5 to about 150 mg/mL, even more preferably about 5 to about 50 mg/mL. When EP₂ agonists are administered in LVLCM or HVLCM vehicles, such as SAIB, multiple doses of such formulations may be required for optimal results. Also, initial administration of the formulations several days after the initial bone fracture, bone injury or bone defect may be preferred.

### EXAMPLE 5

Dissolve 9 g of SAIB in 1 g of ethanol by stirring. Add 0.5 g of EP₂ agonist and stir to suspend or dissolve.

### EXAMPLE 6

Dissolve 8 g of SAIB in 2 g of propylene carbonate by stirring. Add 1 g of EP₂ agonist and stir to suspend or dissolve.

Also, the present invention relates to the use of an intraosseous injectable composition which comprises carbonated apatite (CA) and/or hydroxyapatite and a biocompatible source of calcium for the delivery of locally injected PGE₂ agonists. Sources of calcium ions included, for example calcium sulfate (CS), tricalcium phosphate, monocalcium phosphate and calcium carbonate. The CA or hydroxyapatite may have a particle size of between about 30-3001 μm although a range of about 70-250 μm is preferred. In a particularly preferred form of the invention, the composition comprises 10% to 90% hydroxyapatite, 90% to 10% calcium salt, and up to 20% EP₂ agonist by weight, the balance being distilled water or saline. In a preferred embodiment, the ratio may be 1 part of CA or hydroxyapatite to 3 to 3.5 parts of CS. In the preferred settable composition, 30 to 70%, and preferably 50-60% of the weight of the composition is distilled water; the balance being the solid components.

### EXAMPLE 7

A composition comprising 1 part hydroxyapatite to 3.25 CS, and 5% EP₂ agonist is admixed with approximately 60% distilled water to produce a fine liquid paste.

In addition, the present invention relates to the use of a collagen-containing carrier preparation for the sustained release of locally injected EP₂ agonists (see, e.g., U.S. Pat. No. 4,789,663, which is hereby incorporated by reference, for methods of bone repair using collagen). The carrier will contain at least 5% but preferably at least 10% non-fibrillar collagen and 5-20% EP₂ agonist. The remaining (supplemental) portion of the carrier preparation can be any biocompatible material such as fibrillar collagen, hydroxyapatite, tricalcium phosphate or mixtures thereof. The non-fibrillar (denatured) collagen useful in the invention is used as a solution, as a gel or as a solid, which is non-specifically aggregated after dissolution. The preferred source of non-fibrillar collagen is collagen in solution (CIS). The use of atelopeptide non-fibrillar collagen is preferred, but not required. When EP₂ agonists are administered in collagen-containing carrier preparations, multiple doses of such formulations over a period of time may be required for optimal results to be achieved. Also initial administration of the formulations several days after the initial fracture, injury or defect may be preferred.

Another delivery system which is commercially available and which may be used to formulate the EP₂ agonists of the present invention includes α-BSM™, which is a biomimetic endothermically setting apatitic calcium phosphate bone substitute material developed by ETEX Corporation. It is marketed in Europe by Merck Biomaterial GmBH under the name BioBon®. Another delivery system for formulating the EP₂ agonists of the present invention is Norian®SRS®, which is an injectable calcium phosphate bone cement developed by Norian Corporation. Bone cements in general, including polymethylacrylate (PMMA) cements, may be used to formulate the EP₂ agonists of the present invention. Also, bone glues in general may be used to prepare such formulations. Another commercially available delivery system for formulating the EP₂ agonists of the present invention is BST-Gel® developed by Biosyntech. It is an aqueous-based, ionic polysaccharide gel that is liquid at room temperature and gels at body temperature. In particular, it is based on the polysaccharide chitosan. The EP₂ agonists of the present invention can be incorporated to release slowly at the sites of fracture, injuries or defects in proteins such as thrombin, fibrin or synthetic peptides derived from such proteins.

The advantages of the immediate-release and sustained-release local, preferably injectable, formulations of the EP₂ receptor selective agonists of the present invention include reduction of side effects that often result from oral or systemic administration, such as flushing and diarrhea. The additional advantages of the sustained-release formulations, such as an injectable slow release formulation, may include ensuring a sustained high level of agonist concentration at the local site where the responsible cells are located and perhaps eliminating the multiple injections required for local bone anabolism. Other advantages may include reduction of side effects that result from immediate release formulations, such as irritation at the injection site.

Since the present invention has an aspect that relates to the enhancement of bone repair and healing by treatment with a combination of active ingredients which may be administered separately, the invention also relates to combining separate pharmaceutical compositions in kit form. The kit comprises two separate pharmaceutical compositions: an EP₂ receptor selective compound, a prodrug thereof or a pharmaceutically acceptable salt of said EP₂ receptor selective compound or of said prodrug, and a Second Active Agent, as described above. The kit comprises a container for containing the separate compositions such as a divided bottle or a divided foil packet, however, the separate compositions may also be contained within a single, undivided container. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Evaluation of Test Compounds in Aqueous Solution in the Rat Periosteal Injection Model

I. Rat Periosteal Injection Model

Male Sprague-Dawley rats at 3 weeks of age were used. The rats were anesthetized with isoflurane inhalation (2-3 minutes) in a conduction chamber located in a fume hood. The right hindlimb of each rat was shaved and cleaned. A 26 G needle attached with a hamilton syringe pre-filled with testing solution was used for the local injection. The solution was injected onto the subperiosteum of the anterior, mid-diaphyseal region of the femur in a volume of 5 to 10 ul for various days. On day 15, the rats were sacrificed and the femurs were collected for analysis. Periosteal bone induction was assessed by using radiography, dual-energy X-ray absorptiometry (DEXA) and/or peripheral quantitative computed tomography (pQCT), and histomorphometry. (Michael E, Joyce, Anita B. Roberts, Michael B. Sporn, and Mark Bolander, “Transforming growth factor-β and the initiation of chondrogenesis and osteogenesis in the rat femur,” The Journal of Cell Biology 110:2195-2207 (1990)).

II. Study Protocol and Results

The right femurs of male Spray-Dawley rats were injected with either vehicle or test compound for 1, 3, 7, and 14 days, respectively. The solution was prepared using 2% glycine as vehicle at pH of approximately 7.8-7.9. All rats were sacrificed on day 15 and the right femurs were collected for analysis. One- or three-day treatment with the test compound did not result in periosteal bone formation. Radiography started to show excess calcified mass located on the anterior aspect of the right femur injected with test compound for 7 days. This change became significant after 14 days of treatment. The defined region bone area and bone mineral content (BMC) as assessed by DEXA was significantly increased in the rats treated with the test compound as compared to those treated with vehicle (Table I). The test compound was (3-(((4-tert-butyl-benzyl)-(pyridine-3-sulfonyl)-amino)-methyl)-phenoxy)-acetic acid, sodium salt;

TABLE I

| | Days of | | |
|---|---|---|---|
| Treatment | Dosing | Bone Area (mm²) | BMC (g) |
| Vehicle | 1 | 0.3260 ± 0.0198 | 0.0458 ± 0.0039 |
| Vehicle | 14 | 0.3198 ± 0.0189 | 0.0468 ± 0.0033 |
| CP | 1 | 0.3362 ± 0.0100 | 0.0469 ± 0.0030 |
| CP | 3 | 0.3230 ± 0.0157 | 0.0446 ± 0.0064 |
| CP | 7 | 0.3462 ± 0.0216 | 0.0485 ± 0.0054 |
| CP | 14 | 0.3546 ± 0.0169* | 0.0533 ± 0.0044* |

*Significantly different from 14 days of vehicle treatment
These results demonstrate that these therapeutic regimens are useful in treatment of bone fractures.

III. Study Protocol and Results with Test Compound

The right femur of each rat was injected with test compound for 3, 7 and 14 days, respectively. The left femur of each rat was injected with vehicle to serve as its own control. The solution was prepared using 2% glycine as vehicle at pH of approximately 7.8-7.9. Drug concentration was 80 mg/ml. The injected volume was 5 μl/rat/d (0.4 mg/rat/d). All rats were sacrificed on day 15 and both right and left femurs were collected for analysis. Eight femurs which received 3-day treatment with test compound did not show evidence for increase bone formation locally as assessed by radiography. Two out of eight femurs which received 7-day treatment with test compound started to show increased calcified area. All femurs (n=8) which received 14-day treatment with test compound showed increased calcified area locally as compared to controls. The test compound was 7-{[2-(3,5-dichloro-phenoxy)-ethyl]-methanesulfonyl-amino}-heptanoic acid.

### Enhancement of Bone Healing in Dog Model

It is clinically complex to heal segmental bone loss and non-union after fractures or reconstructive surgery. In recent years, bone morphogenic proteins (BMP's) have been extensively tested in various pre-clinical models of segmental defects that do not heal spontaneously if left untreated. These models have proved to be extremely important in characterizing the osteoinductive abilities of BMP's and other bone inductive agents. The following is a description of the ulnar segmental defect model used to evaluate bone healing in 11±1 kg beagle male dogs, 13 months old.

Beagle dogs were treated with antiparasitics one week before surgery and were given two doses of Baypamun (Bayer), 72 and 24 hours before the operation. Dogs were divided into four groups of eight animals.

Group A: 2 ml of phosphate buffered saline (PBS) was injected into the defect area filled with two helistat pre-cut sponges (HELISTAT; 2.5×5 cm) 24, 48 and 72 hours following surgery.

Group B: 100 mg preparation of test compound was injected into the defect area filled with two helistat pre-cut sponges (HELISTAT; 2.5×5 cm) 24 hours following surgery and for three consecutive days (24, 48 and 72 hours).

Group C: 100 mg preparation of test compound was injected into the defect area filled with two helistat pre-cut sponges (HELISTAT; 2.5×5 cm) beginning 24 hours following surgery and daily thereafter for seven consecutive days.

Group D: 100 mg preparation of test compound was injected into the defect area filled with two helistat pre-cut sponges (HELISTAT; 2.5×5 cm) 24 hours following surgery and daily thereafter for 14 days.

The test compound was (3-(((4-tert-butyl-benzyl)-(pyridine-3-sulfonyl)-amino)-methyl)-phenoxy)-acetic acid, sodium salt.

With animals under general anesthesia, the foreleg was prepped and draped in sterile fashion. A lateral incision approximately 10 cm in length was made and the ulna was exposed extraperiostally. Periosteum was cut and moved to the proximal and distal parts of the incision. Then 1.5 cm segmental defect was made in the midulna using a pendular saw. The radius and the remaining interosseal membrane were left intact. The defect site was irrigated with saline to remove bone debris. Bone fixation was achieved with two 2.0 mm cortical screws placed approximately 1.5 cm away from the defect ends not to compromise healing and subsequent dermal injections. Both created bone ends were firmly stable and the radius served as a weight-bearing bone during the recovery process.

The site was then filled with the two helistat sponges as described above. In particular two Helistat sponges sized 2.5 times 5 cm were rolled to form a cylinder which was secured outside with fibrin net and two resorbable sutures. In that way subsequent dermal injections had larger areas of absorption. The soft tissues were meticulously closed in layers to aid the sponges. Injections were administered via dermal suture markers in such a way that the needle reached the opposing radius and then pulled back for approximately 5 mm. The total volume per injection was 2 ml consisting either of vehicle (PBS) or test compound in PBS. Following surgery animals were allowed full weight-bearing activity, water and food ad libitum. Radiographs of the forelimbs were obtained immediately following surgery and every two weeks thereafter until the termination of the study. Radiographs were graded on a 0 to 6 scale (Table A).

TABLE A
Radiographic Grading Scale

| | |
|---|---|
| Grade 0 | no change from immediate postoperative appearance |
| Grade 1 | trace of radiodense material in defects |
| Grade 2 | flocculent radiodensity with flecks of calcification and no defect |
| | bridging |
| Grade 3 | defect bridged at least one point with material of nonuniform |
| | radiodensity |
| Grade 4 | defect bridged in medial and lateral sides with material of |
| | uniform radiodensity, cut ends of cortex remain visible |
| Grade 5 | same as Grade 3, at least one of four cortices obscured by new |
| | bone |
| Grade 6 | defect bridged by uniform new bone, cut ends of cortex not seen |

Dogs were sacrificed 12 weeks following surgery and the ulna was carefully dissected and fixed in 10% buffered formalin for histological analysis. As expected none of the control dogs re-bridged the defect confirming that the defect was a critical sized defect (Table B). Moreover, the non-union was pronounced permanent at the termination of the study since no significant progress in radiographs was observed between four and twelve weeks following surgery. In the 3-injections groups, none of the defects were re-bridged at the termination of the study. However, new bone induction was observed in all the dogs as a result of both osteoconduction and periosteal reaction. One dog also showed bone formation in the middle of the defect that was not connected to the bone ends. Histological analysis confirmed complete mineralization of the newly formed bone. Radiographic scoring indicated a score between 2 and three for the dogs in this group.

In the 7-injections group, similar to the 3-injections group, none of the dogs showed a full re-bridgement. Both endosteal and periosteal bone formation was observed in the defect area. Radiographic scoring showed that scores were as high as 4 in one of the dogs. Histological analysis confirmed that newly formed bone was fully mineralized and there was no evidence of cartilage anlage suggesting that bone formation had been accomplished.

In the final, 14-injections group, two of the eight dogs showed full re-bridgement by both x-rays and histology. Both the animals showed a well shaped newly formed bone which was fused with both ulnar bone ends. Three other dogs showed a large amount of new bone formation in the defect area and the surrounding periosteum but did not completely fill the defect. Three dogs showed relatively less bone formation and were pronounced non-responders. The major reason for this could be the relatively non-controlled application of the test compound. Histological analysis of the healed bone revealed that the new bone consisted of dense trabeculi covered with osteoid seams and active bone cells both osteoblasts and osteoclasts. There was also a well developed bone marrow between the newly formed bone.

TABLE B
Results

| | Radiographic Grading/8 | |
|---|---|---|
| Groups | dogs | Bridging/8 dogs |
| A | 1-2 | 1 dog showed signs of medial |
| | | bridging |
| B | 1-3 | 0/8 |
| C | 2-4 | 1/8 showed medial healing |
| D | 4-6 | 4/8 showed good bridging. Three |
| | | showed almost complete healing |

1. A method for treating a bone fracture, bone injury or bone defect in a patient comprising local administration to the patient of a therapeutically effective amount of an EP₂ receptor selective agonist once a day for a period of about 7 days or greater.

2. A method of claim 1 wherein the agonist is administered once a day for about 7 to about 14 days.

3. A method of claim 1 wherein the agonist is administered once a day for about 14 days.

4. A method of claim 1 wherein the agonist is administered once a day for about 14 to about 21 days.

5. A method of claim 1 wherein the agonist is administered once a day for about 14 to about 28 days.

6. A method of claim 1 wherein the therapeutically effective amount of the agonist is between about 0.001 to about 100 mg/kg/day

7. A method of claim 6 wherein the amount of the agonist is between about 0.01 to about 10 mg/kg/day.

8. A method of claim 1 wherein the agonist is administered by direct injection in a pharmaceutically acceptable buffer at or near the site where bone growth is needed.

9. A method of claim 8 wherein the agonist is administered by direct injection in a pharmaceutically acceptable buffer at or near the site of the bone fracture, bone injury or bone defect.

10. A method of claim 1 wherein the agonist is administered by a catheter at or near the site where bone growth is needed.

11. A method for treating a bone fracture, bone injury or bone defect in a patient comprising local administration to the patient of a therapeutically effective amount of an EP₂ receptor selective agonist in a controlled release formulation;
wherein the agonist is administered in an oily suspension of an insoluble salt of the agonist;
wherein the agonist is administered in a bone glue formulation;
wherein the agonist is administered in a hydrophilic matrix containing poloxamers;
wherein the agonist is administered in controlled-release, biodegradable lipid vessicles;
wherein the agonist is administered in controlled-release, biodegradable poly(lactide-co-glycolide) microparticles;
wherein the agonist is administered in a polyanionic polysaccharide formulation;
wherein the agonist is administered in high viscosity liquid carrier material or lower viscosity liquid carrier material;
wherein the agonist is administered in carbonated apatite or hydroxyapatite formulation and a biocompatible source of calcium;
wherein the agonist is administered in collagen-containing carrier preparation; or
wherein the agonist is administered in formulations of thrombin, fibrin or synthetic peptides derived therefrom.

12. A method of claim 11 wherein the lipid vessicles are liposomes.

13. A method of claim 11 wherein the polyanionic polysaccharide is hyaluronic acid or carboxymethylcellulose.

14. A method of claim 11 wherein the high viscosity liquid carrier material is sucrose acetate isobutyrate.

15. A method of claim 11 wherein the agonist is released for a period of about 3 days or greater.

16. A method of claim 15 wherein the agonist is released over a period of about 7 to about 28 days.

17. A method of claim 16 wherein the agonist is released over a period of about 7 to about 14 days.

18. A method of claim 17 wherein the agonist is released over a period of about 12 to about 14 days.

19. A method of claim 11 wherein the agonist is administered by direct injection at or near the site where bone growth is needed.

20. A method of claim 19 wherein the agonist is administered by direct injection at or near the site of the bone fracture, bone injury or bone defect.

21. A method of claim 1 or 11 wherein the EP₂ receptor selective agonist is a compound of Formula I 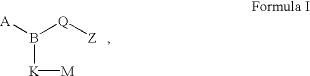
a prodrug thereof, or a pharmaceutically acceptable salt of the compound or the prodrug, wherein:
B is N;
A is (C₁-C₆)alkylsulfonyl, (C₃-C₇)cycloalkylsulfonyl, (C₃-C₇)cycloalkyl(C₁-C₆)alkylsulfonyl, said A moieties optionally mono-, di- or tri-substituted on carbon independently with hydroxy, (C₁-C₄)alkyl or halo;
Q is
—(C₂-C₆)alkylene-W—(C₁-C₃)alkylene-,
—(C₃-C₈)alkylene-, said —(C₃-C₈)alkylene- optionally substituted with up to four substituents independently selected from fluoro or (C₁-C₄)alkyl,
—X—(C₁-C₅)alkylene-,
—(C₁-C₅)alkylene-X—,
—(C₁-C₃)alkylene-X—(C₁-C₃)alkylene-,
—(C₂-C₄)alkylene-W-X—(CO—C₃)alkylene-,
—(CO—C₄)alkylene-X—W—(C₁-C₃)alkylene-,
—(C₂-C₅)alkylene-W—X—W—(C₁-C₃)alkylene-, wherein the two occurrences of W are independent of each other,
—(C₁-C₄)alkylene-ethenylene-(C₁-C₄)alkylene-,
—(C₁-C₄)alkylene-ethenylene-(CO—C₂)alkylene-X—(CO—C₅)alkylene-,
—(C₁-C₄)alkylene-ethenylene-(CO—C₂)alkylene-X—W—(C₁-C₃)alkylene-,
—(C₁-C₄)alkylene-ethynylene-(C₁-C₄)alkylene-, or
—(C₁-C₄)alkylene-ethynylene-X—(CO—C₃)alkylene-;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five- or six-membered aromatic ring optionally having one or two heteroatoms selected independently from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;
Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;
K is a bond, (C₁-C₈)alkylene, thio(C₁-C₄)alkylene or oxy(C₁-C₄)alkylene, said (C₁-C₈)alkylene optionally mono-unsaturated and wherein K is optionally mono-, di- or tri-substituted independently with fluoro, methyl or chloro;
M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five- to eight-membered ring optionally having one to four heteroatoms selected independently from oxygen, sulfur and nitrogen, or, a bicyclic ring consisting of two fused partially saturated, fully saturated or fully unsaturated five- or six-membered rings, taken independently, optionally having one to four heteroatoms selected independently from nitrogen, sulfur and oxygen;
said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
R¹, R² and R³ are optionally mono-, di- or tri-substituted on carbon independently with halo or hydroxy; and
V is a bond or (C₁-C₃)alkylene optionally mono- or di-substituted independently with hydroxy or fluoro;

22. A method of claim 21 where the compound of Formula I is selected from the group consisting of:
7-[(2′-hydroxymethyl-biphenyl-4-ylmethyl)-methanesulfonyl-amino]-heptanoic acid;
7-{[4-(3-hydroxymethyl-thiophen-2-yl)-benzyl]-methanesulfonyl-amino}-heptanoic acid;
7-[(2′-chloro-biphenyl-4-ylmethyl)-methanesulfonyl-amino]-heptanoic acid;
7-{[4-(1-hydroxy-hexyl)-benzyl]-methanesulfonyl-amino}-heptanoic acid;
7-[(4-butyl-benzyl)-methanesulfonyl-amino]-heptanoic acid;
7-{[5-(1-hydroxy-hexyl)-thiophen-2-ylmethyl]-methanesulfonyl-amino}-heptanoic acid;
(3-{[(4-butyl-benzyl)-methanesufonyl-amino]-methyl}-phenyl)-acetic acid;
7-{[3-(3-chloro-phenyl)-propyl]-methanesulfonyl-amino}-heptanoic acid;
7-{[3-(3,5-dichloro-phenyl)-propyl]-methanesufonyl-amino}-heptanoic acid;
5-(3-{[3-(3-chloro-phenyl)-propyl]-methanesulfonyl-amino}-propyl)-thiophene-2-carboxylic acid;
7-{[2-(3,5-dichloro-phenoxy)-ethyl]-methanesulfonyl-amino}-heptanoic acid;
5-(3-{[2-(3,5-dichloro-phenoxy)-ethyl]-methanesulfonyl-amino}-propyl)-thiophene-2-carboxylic acid;
N-[2-(3,5-dichloro-phenoxy)-ethyl]-N-[6-(1H-tetrazol-5-yl)-hexyl]-methanesulfonamide;
trans-(4-{[3-(3,5-dichloro-phenyl)-allyl]-methanesulfonyl-amino}-butoxy)-acetic acid;
trans-N-[3-(3,5-dichloro-phenyl)-allyl]-N-[6-(1H-tetrazol-5-yl)-hexyl]-methanesulfonamide;
trans-5-(3-{[3-(3,5-dichloro-phenyl)-allyl]-methanesulfonyl-amino}-propyl)-thiophene-2-carboxylic acid; and
trans-[3-({[3-(3,5-dichloro-phenyl)-allyl]-methanesulfonyl-amino}-methyl)-phenyl]-acetic acid; a prodrug thereof, or a pharmaceutically acceptable salt of the compound or the prodrug.

23. A method of claim 22 wherein the EP₂ receptor selective agonist is 7-[(4-butyl-benzyl)-methanesulfonyl-amino]-heptanoic acid; or
7-{[2-(3,5-dichloro-phenoxy)-ethyl]-methanesulfonyl-amino}-heptanoic acid; or a pharmaceutically acceptable salt thereof.

24. A method of claim 1 or 11 wherein the EP₂ receptor selective agonist is a compound of Formula II 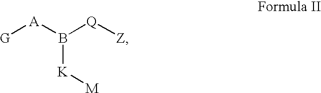
a prodrug thereof, or a pharmaceutically acceptable salt of the compound or the prodrug, wherein:
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—(CO—C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X-W—(C₁-C₃)alkylene-, said alkylenes and said ethenylene each optionally substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, taken independently, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five- to seven-membered ring having one or two heteroatoms selected independently from oxygen, sulfur and nitrogen;
Ar¹ and Ar² are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five- or six-membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethoxy, difluoromethoxy, hydroxyl, (C₁-C₄)alkoxy, or carbamoyl;
R¹, R², R³, R⁴ R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro.

25. A method of claim 24 wherein the compound of Formula II is selected from the group consisting of:
(3-(((pyridine-3-sulfonyl)-(4-pyrimidin-5-yl-benzyl)-amino)-methyl)-phenyl)-acetic acid;
(3-(((5-phenyl-furan-2-ylmethyl)-(pyridine-3-sulfonyl)-amino)-methyl)-phenyl)-acetic acid;
(3-(((pyridine-3-sulfonyl)-(4-pyrimidin-2-yl-benzyl)-amino)-methyl)-phenyl)-acetic acid;
(3-(((pyridine-3-sulfonyl)-(4-thiazol-2-yl-benzyl)-amino)-methyl)-phenyl)-acetic acid;
(3-(((4-pyrazin-2-yl-benzyl)-(pyridine-3-sulfonyl)-amino)-methyl)-phenyl)-acetic acid;
(3-(((4-cyclohexyl-benzyl)-(pyridine-3-sulfonyl)-amino)-methyl)-phenoxy)-acetic acid;
(3-(((pyridine-3-sulfonyl)-(4-pyridin-2-yl-benzyl)-amino)-methyl)-phenoxy)-acetic acid;
(3-(((pyridine-3-sulfonyl)-(4-pyridin-3-yl-benzyl)-amino)-methyl)-phenoxy)-acetic acid;
(3-(((pyridine-3-sulfonyl)-(4-pyridin-4-yl)-benzyl)-amino)-methyl)-phenoxy)-acetic acid;
(3-(((pyridine-3-sulfonyl)-(4-thiazol-2-yl-benzyl)-amino)-methyl)-phenoxy)-acetic acid;
(3-(((2,3-dihydro-benzo[1,4]dioxin-6-ylmethyl)-(pyridine-3-sulfonyl)-amino)-methyl)-phenyl)-acetic acid;
(3-(((benzofuran-2-ylmethyl-(pyridine-3-sulfonyl)-amino)-methyl)-phenyl)-acetic acid;
(3-(((4-butyl-benzyl)-(pyridine-3-sulfonyl)-amino)-methyl)-phenyl)-acetic acid;
(3-(((benzenesulfonyl-(4-butyl-benzyl)-amino)-methyl)-phenyl)-acetic acid;
(3-(((4-butyl-benzyl)-(1-methyl-1H-imidazole-4-sulfonyl)-amino)-methyl)-phenyl)-acetic acid;
(3-(((4-dimethylamino-benzyl)-(pyridine-3-sulfonyl)-amino)-methyl)-phenyl)-acetic acid;
(3-(((4-dimethylamino-benzyl)-(pyridine-3-sulfonyl)-amino)-methyl)-phenoxy)-acetic acid;
(3-(((4-tert-butyl-benzyl)-(pyridine-3-sulfonyl)-amino)-methyl)-phenoxy)-acetic acid;
trans-(3-(((3-(3,5-dichloro-phenyl)-allyl)-(pyridine-3-sulfonyl)-amino)-methyl)-phenyl)-acetic acid; and
(3-(((2-(3,5-dichloro-phenoxy)-ethyl)-(pyridine-3-sulfonyl)-amino)-methyl)-phenoxy)-acetic acid; a prodrug thereof, or a pharmaceutically acceptable salt of the compound or the prodrug.

26. A method of claim 25 wherein the EP₂ receptor selective agonist is the sodium salt of (3-(((4-tert-butyl-benzyl)-(pyridine-3-sulfonyl)-amino)-methyl)-phenoxy)-acetic acid.

27. A controlled release microparticle pharmaceutical composition for the sustained release of an EP₂ receptor selective agonist which comprises an EP₂ receptor selective agonist and a biocompatible, biodegradable poly(lactide-co-glycolide) polymer.

28. A composition of claim 27 wherein the EP₂ receptor selective agonist is a compound of Formula I 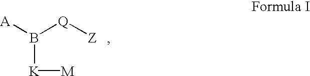
a prodrug thereof, or a pharmaceutically acceptable salt of the compound or the prodrug, wherein:
B is N;
A is (C₁-C₆)alkylsulfonyl, (C₃-C₇)cycloalkylsulfonyl, (C₃-C₇)cycloalkyl(C₁-C₆)alkylsulfonyl, said A moieties optionally mono-, di- or tri-substituted on carbon independently with hydroxy, (C₁-C₄)alkyl or halo;
Q is
—(C₂-C₆)alkylene-W—(C₁-C₃)alkylene-,
—(C₃-C₈)alkylene-, said —(C₃-C₈)alkylene- optionally substituted with up to four substituents independently selected from fluoro or (C₁-C₄)alkyl,
—X—(C₁-C₅)alkylene-,
—(C₁-C₅)alkylene-X—,
—(C₁-C₃)alkylene-X—(C₁-C₃)alkylene-,
—(C₂-C₄)alkylene-W-X—(CO—C₃)alkylene-,
—(C₀-C₄)alkylene-X-W—(C₁-C₃)alkylene-,
—(C₂-C₅)alkylene-W-X-W—(C₁-C₃)alkylene-, wherein the two occurrences of W are independent of each other,
—(C₁-C₄)alkylene-ethenylene-(C₁-C₄)alkylene-,
—(C₁-C₄)alkylene-ethenylene-(CO—C₂)alkylene-X—(C₀-C₅)alkylene-,
—(C₁-C₄)alkylene-ethenylene-(C₀-C₂)alkylene-X-W—(C₁-C₃)alkylene-,
—(C₁-C₄)alkylene-ethynylene-(C₁-C₄)alkylene-, or
—(C₁-C₄)alkylene-ethynylene-X—(C₀-C₃)alkylene-;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five- or six-membered aromatic ring optionally having one or two heteroatoms selected independently from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;
Z is carboxyl, (C₁-C₆)alkoxycarbonyl, tetrazolyl, 1,2,4-oxadiazolyl, 5-oxo-1,2,4-oxadiazoiyl, (C₁-C₄)alkylsulfonylcarbamoyl or phenylsulfonylcarbamoyl;
K is a bond, (C₁-C₈)alkylene, thio(C₁-C₄)alkylene or oxy(C₁-C₄)alkylene, said (C₁-C₈)alkylene optionally mono-unsaturated and wherein K is optionally mono-, di- or tri-substituted independently with fluoro, methyl or chloro;
M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five- to eight-membered ring optionally having one to four heteroatoms selected independently from oxygen, sulfur and nitrogen, or, a bicyclic ring consisting of two fused partially saturated, fully saturated or fully unsaturated five- or six-membered rings, taken independently, optionally having one to four heteroatoms selected independently from nitrogen, sulfur and oxygen;
said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
R¹, R² and R³ are optionally mono-, di- or tri-substituted on carbon independently with halo or hydroxy; and
V is a bond or (C₁-C₃)alkylene optionally mono- or di-substituted independently with hydroxy or fluoro;

29. A composition of claim 28 where the compound of Formula I is selected from the group consisting of:
7-[(2′-hydroxymethyl-biphenyl-4-ylmethyl)-methanesulfonyl-amino]-heptanoic acid;
7-{[4-(3-hydroxymethyl-thiophen-2-yl)-benzyl]-methanesulfonyl-amino}-heptanoic acid;
7-[(2′-chloro-biphenyl-4-ylmethyl)-methanesulfonyl-amino]-heptanoic acid;
7-{[4-(1-hydroxy-hexyl)-benzyl]-methanesulfonyl-amino}-heptanoic acid;
7-[(4-butyl-benzyl)-methanesulfonyl-amino]-heptanoic acid;
7-{[5-(1-hydroxy-hexyl)-thiophen-2-ylmethyl]-methanesulfonyl-amino}-heptanoic acid;
(3-{[(4-butyl-benzyl)-methanesufonyl-amino]-methyl}-phenyl)-acetic acid;
7-{[3-(3-chloro-phenyl)-propyl]-methanesulfonyl-amino}-heptanoic acid;
7-{[3-(3,5-dichloro-phenyl)-propyl]-methanesufonyl-amino}-heptanoic acid;
5-(3-{[3-(3-chloro-phenyl)-propyl]-methanesulfonyl-amino}-propyl)-thiophene-2-carboxylic acid;
7-{[2-(3,5-dichloro-phenoxy)-ethyl]-methanesulfonyl-amino}-heptanoic acid;
5-(3-{[2-(3,5-dichloro-phenoxy)-ethyl]-methanesulfonyl-amino}-propyl)-thiophene-2-carboxylic acid;
N-[2-(3,5-dichloro-phenoxy)-ethyl]-N-[6-(1H-tetrazol-5-yl)-hexyl]-methanesulfonamide;
trans-(4-{[3-(3,5-dichloro-phenyl)-allyl]-methanesulfonyl-amino}-butoxy)-acetic acid;
trans-N-[3-(3,5-dichloro-phenyl)-allyl]-N-[6-(1H-tetrazol-5-yl)-hexyl]-methanesulfonamide;
trans-5-(3-{[3-(3,5-dichloro-phenyl)-allyl]-methanesulfonyl-amino}-propyl)-thiophene-2-carboxylic acid; and
trans-[3-({[3-(3,5-dichloro-phenyl)-allyl]-methanesulfonyl-amino}-methyl)-phenyl]-acetic acid; a prodrug thereof, or a pharmaceutically acceptable salt of the compound or the prodrug.

30. A composition of claim 29 wherein the EP₂ receptor selective agonist is 7-[(4-butyl-benzyl)-methanesulfonyl-amino]-heptanoic acid; or
7-{[2-(3,5-dichloro-phenoxy)-ethyl]-methanesulfonyl-amino}-heptanoic acid; or a pharmaceutically acceptable salt thereof.

31. A composition of claim 27 wherein the EP₂ receptor selective agonist is a compound of Formula II 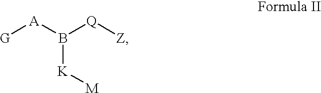
a prodrug thereof, or a pharmaceutically acceptable salt of the compound or the prodrug, wherein:
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
—(C₂-C₆)alkylene-W—(C₁-C₃)alkylene-, said alkylenes each optionally substituted with up to four substituents independently selected from fluoro or (C₁-C₄)alkyl,
—(C₄-C₈)alkylene-, said alkylene optionally substituted with up to four substituents independently selected from fluoro or (C₁-C₄)alkyl,
—X—(C₁-C₅)alkylene-, said alkylene optionally substituted with up to four substituents independently selected from fluoro or (C₁-C₄)alkyl,
—(C₁-C₅)alkylene-X—, said alkylene optionally substituted with up to four substituents independently selected from fluoro or (C₁-C₄)alkyl,
—(C₁-C₃)alkylene-X—(C₁-C₃)alkylene-, said alkylenes each optionally substituted with up to four substituents independently selected from fluoro or (C₁-C₄)alkyl,
—(C₂-C₄)alkylene-W—X—(C₀-C₃)alkylene-, said alkylenes each optionally substituted with up to four substituents each independently selected from fluoro or (C₁-C₄)alkyl,
—(C₀-C₄)alkylene-X—W—(C₁-C₃)alkylene-, said alkylenes each optionally substituted with up to four substituents each independently selected from fluoro or (C₁-C₄)alkyl,
—(C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
—(C₁-C₄)alkylene-ethenylene-(C₁-C₄)alkylene-, said alkylenes and said ethenylene each optionally substituted with up to four substituents each independently selected from fluoro or (C₁-C₄)alkyl,
—(C₁-C₄)alkylene-ethenylene-(CO—C₂)alkylene-X—(C₀-C₅)alkylene-, said alkylenes and said ethenylene each optionally substituted with up to four substituents each independently selected from fluoro or (C₁-C₄)alkyl,
—(C₁-C₄)alkylene-ethenylene-(C₀-C₂)alkylene-X—W—(C₁-C₃)alkylene-, said alkylenes and said ethenylene each optionally substituted with up to four substituents each independently selected from fluoro or (C₁-C₄)alkyl,
—(C₁-C₄)alkylene-ethynylene-(C₁-C₄)alkylene-, said alkylenes and said ethynylene each optionally substituted with up to four substituents each independently selected from fluoro or (C₁-C₄)alkyl, or
—(C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³—Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, taken independently, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five- to seven-membered ring having one or two heteroatoms selected independently from oxygen, sulfur and nitrogen;
Ar¹ and Ar² are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
Ar³, Ar⁴ and Ar⁵ are each independently a partially saturated, fully saturated or fully unsaturated five- to eight-membered ring optionally having one to four heteroatoms selected independently from oxygen, sulfur and nitrogen, or a bicyclic ring consisting of two fused independently partially saturated, fully saturated or fully unsaturated five- or six-membered rings, taken independently, optionally having one to four heteroatoms selected independently from nitrogen, sulfur and oxygen, or a tricyclic ring consisting of three fused independently partially saturated, fully saturated or fully unsaturated five- or six-membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N—, di-N,N—, di-N,N′— or tri-N,N,N′—(C₁-C₄)alkyl substituted aminocarbonylamino, sulfonamido, (C₁-C₄)alkylsulfonamido, amino, mono-N— or di-N,N—(C₁-C₄)alkylamino, carbamoyl, mono-N— or di-N,N—(C₁-C₄)alkylcarbamoyl, cyano, thiol, (C₁-C₆)alkylthio, (C₁-C₆)alkylsulfinyl, (C₁-C₄)alkylsulfonyl or mono-N— or di-N,N—(C₁-C₄)alkylaminosulfinyl;
W is oxy, thio, sulfino, sulfonyl, aminosulfonyl-, -mono-N—(C₁-C₄)alkyleneaminosulfonyl-, sulfonylamino, N—(C₁-C₄)alkylenesulfonylamino, carboxamido, N—(C₁-C₄)alkylenecarboxamido, carboxamidooxy, N—(C₁-C₄)alkylenecarboxamidooxy, carbamoyl, -mono-N—(C₁-C₄)alkylenecarbamoyl, carbamoyloxy, or -mono-N—(C₁-C₄)alkylenecarbamoyloxy, wherein said W alkyl groups are optionally substituted on carbon with one to three fluorines;
X is a five- or six-membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethoxy, difluoromethoxy, hydroxyl, (C₁-C₄)alkoxy, or carbamoyl;
R¹, R², R³, R⁴, R⁵ R¹¹, R³¹, R⁴¹ and R⁵¹, when containing an alkyl, alkylene, alkenylene or alkynylene moiety, are optionally mono-, di- or tri-substituted on carbon independently with halo or hydroxy; and
V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro.

32. A composition of claim 31 wherein the compound of Formula II is selected from the group consisting of:
(3-(((pyridine-3-sulfonyl)-(4-pyrimidin-5-yl-benzyl)-amino)-methyl)-phenyl)-acetic acid;
(3-(((5-phenyl-furan-2-ylmethyl)-(pyridine-3-sulfonyl)-amino)-methyl)-phenyl)-acetic acid;
(3-(((pyridine-3-sulfonyl)-(4-pyrimidin-2-yl-benzyl)-amino)-methyl)-phenyl)-acetic acid;
(3-(((pyridine-3-sulfonyl)-(4-thiazol-2-yl-benzyl)-amino)-methyl)-phenyl)-acetic acid;
(3-(((4-pyrazin-2-yl-benzyl)-(pyridine-3-sulfonyl)-amino)-methyl)-phenyl)-acetic acid;
(3-(((4-cyclohexyl-benzyl)-(pyridine-3-sulfonyl)-amino)-methyl)-phenoxy)-acetic acid;
(3-(((pyridine-3-sulfonyl)-(4-pyridin-2-yl-benzyl)-amino)-methyl)-phenoxy)-acetic acid;
(3-(((pyridine-3-sulfonyl)-(4-pyridin-3-yl-benzyl)-amino)-methyl)-phenoxy)-acetic acid;
(3-(((pyridine-3-sulfonyl)-(4-pyridin-4-yl)-benzyl)-amino)-methyl)-phenoxy)-acetic acid;
(3-(((pyridine-3-sulfonyl)-(4-thiazol-2-yl-benzyl)-amino)-methyl)-phenoxy)-acetic acid;
(3-(((2,3-dihydro-benzo[1,4]dioxin-6-ylmethyl)-(pyridine-3-sulfonyl)-amino)-methyl)-phenyl)-acetic acid;
(3-(((benzofuran-2-ylmethyl-(pyridine-3-sulfonyl)-amino)-methyl)-phenyl)-acetic acid;
(3-(((4-butyl-benzyl)-(pyridine-3-sulfonyl)-amino)-methyl)-phenyl)-acetic acid;
(3-(((benzenesulfonyl-(4-butyl-benzyl)-amino)-methyl)-phenyl)-acetic acid;
(3-(((4-butyl-benzyl)-(1-methyl-1H-imidazole-4-sulfonyl)-amino)-methyl)-phenyl)-acetic acid;
(3-(((4-dimethylamino-benzyl)-(pyridine-3-sulfonyl)-amino)-methyl)-phenyl)-acetic acid;
(3-(((4-dimethylamino-benzyl)-(pyridine-3-sulfonyl)-amino)-methyl)-phenoxy)-acetic acid;
(3-(((4-tert-butyl-benzyl)-(pyridine-3-sulfonyl)-amino)-methyl)-phenoxy)-acetic acid;
trans-(3-(((3-(3,5-dichloro-phenyl)-allyl)-(pyridine-3-sulfonyl)-amino)-methyl)-phenyl)-acetic acid; and
(3-(((2-(3,5-dichloro-phenoxy)-ethyl)-(pyridine-3-sulfonyl)-amino)-methyl)-phenoxy)-acetic acid; a prodrug thereof, or a pharmaceutically acceptable salt of the compound or the prodrug.

33. A composition of claim 32 wherein the EP₂ receptor selective agonist is the sodium salt of (3-(((4-tert-butyl-benzyl)-(pyridine-3-sulfonyl)-amino)-methyl)-phenoxy)-acetic acid.

34. A composition of claim 27 wherein the composition is locally administered at or near the site of the bone fracture, bone injury or bone defect.

35. A composition of claim 27 wherein the agonist is released over a period of about 7 to about 28 days.
